(19) United States
(12) Patent Application Publication
(10) Pub. No.: US 2025/0225377 A1
(43) Pub. Date: Jul. 10, 2025

(54) GENERATING LARGE-LANGUAGE-MODEL COMPATIBLE SEQUENTIAL ATTACHMENT-BASED FRAGMENT EMBEDDING MOLECULAR REPRESENTATIONS

(71) Applicant: Recursion Pharmaceuticals, Inc., Salt Lake City, UT (US)

(72) Inventors: Cristian GABELLINI, Montreal (CA); Finagnon Marc-Rolland Emmanuel NOUTAHI, Montreal (CA); Michael Murphy CRAIG, Montreal (CA); Prudencio Murphy Akouété TOSSOU, Montreal (CA)

(21) Appl. No.: 18/750,832

(22) Filed: Jun. 21, 2024

Related U.S. Application Data
(60) Provisional application No. 63/618,172, filed on Jan. 5, 2024.

(51) Int. Cl. G06N 3/0455; G06F 40/284; G16C 20/50

(52) U.S. Cl. CPC G06N 3/0455; G06F 40/284; G16C 20/50

(57) ABSTRACT

The present disclosure relates to systems, non-transitory computer-readable media, and methods for generating a sequential attachment-based fragment embedding (SAFE) molecular string representation that represents a molecular representation as an order agnostic sequence of interconnected fragment blocks. Indeed, the disclosed systems can generate the SAFE representation for processing via large language models for downstream molecular design tasks. For instance, the disclosed systems can extract fragments (and attachment points) from a molecular string representation, concatenate the extracted fragments using separation character connections between the fragments to generate a set of linked fragments, and can iterate over attachment points for the fragments to generate ring link characters in the set of linked fragments to simulate fragment links. In addition, the disclosed systems can utilize the SAFE representation to enable various downstream fragment-based molecular design tasks via large language models.

### CROSS-REFERENCE TO RELATED APPLICATIONS

This application claims priority to and the benefit of U.S. Provisional Patent Application No. 63/618,172, filed on Jan. 5, 2024, which is incorporated herein by reference in its entirety.

### BACKGROUND

Recent years have seen significant improvements in hardware and software platforms for molecular design in computational drug discovery. In particular, existing systems often utilize computing devices and corresponding models to construct molecules with desired characteristics. In addition, existing systems often preserve certain scaffolds or core chemical substructures that serve as the backbone for the computer-based molecular design process because these scaffolds and constraints are crucial to a molecule's biological activity. In many cases, existing systems utilize a molecular string representation, Simplified Molecular Input Line Entry System (SMILES), within a drug discovery system. Although existing systems utilize molecular string representations, such as SMILES, existing systems often have a number of technical shortcomings with regard to the flexibility and accuracy that limit artificial intelligent (AI)-driven molecular design tasks.

### SUMMARY

Embodiments of the present disclosure provide benefits and/or solve one or more of the foregoing or other problems in the art with systems, non-transitory computer-readable media, and computer-implemented methods for generating a sequential attachment-based fragment embedding (SAFE) molecular string representation that represents a molecular representation as an order agnostic sequence of interconnected fragment blocks. Indeed, the disclosed systems can generate the SAFE molecular string representation for processing via large language models for downstream molecular design tasks. For instance, the disclosed systems can extract fragments (and attachment points) from a molecular string representation (e.g., a SMILES molecular string representation). Moreover, the disclosed systems can concatenate the extracted fragments using separation character connections between the fragments to generate a set of linked fragments (e.g., as a string). In addition, the disclosed systems can iterate over attachment points for the fragments to generate ring link characters in the set of linked fragments to simulate fragment links. Indeed, the resulting SAFE molecular string representation can include an order agnostic sequence of interconnected fragment blocks that represent a molecular compound.

Furthermore, the disclosed systems can utilize the above-mentioned SAFE molecular string representation to enable various downstream fragment-based molecular design tasks via machine learning models (that are not viable using many existing molecular representation notations). For instance, the disclosed systems can train a large language model for the fragment-based molecular design tasks by training the large language model via a measure of loss between a predicted completion of a partial sequence of a training SAFE molecular string representation and the training SAFE molecular string representation. Indeed, the SAFE molecular representation large language model can be utilized for de novo molecular compound generation tasks, scaffold decoration and motif extension tasks, linker design and scaffold morphing tasks, and/or molecular superstructure generation tasks.

Additional features and advantages of one or more embodiments of the present disclosure are outlined in the description which follows, and in part can be determined from the description, or may be learned by the practice of such example embodiments.

### BRIEF DESCRIPTION OF THE DRAWINGS

The detailed description is described with reference to the accompanying drawings in which:

FIG. 1 illustrates an overview of a digital molecular structure generation system generating and utilizing SAFE molecular string representations in accordance with one or more embodiments.

FIG. 2 illustrates a digital molecular structure generation system converting a molecular string representation into a SAFE molecular string representation in accordance with one or more embodiments.

FIG. 3 illustrates a flow diagram of a digital molecular structure generation system generating SAFE molecular string representations in accordance with one or more embodiments.

FIG. 4 illustrates a digital molecular structure generation system utilizing SAFE molecular string representations to train a SAFE generative model in accordance with one or more embodiments.

FIG. 5 illustrates a digital molecular structure generation system utilizing a SAFE generative model to perform various downstream fragment-based molecular design tasks in accordance with one or more embodiments.

FIGS. 6-12 illustrate experimental results of implementations of a SAFE generative model in accordance with one or more embodiments.

FIG. 13 illustrates a schematic diagram of a system environment in which a digital molecular structure generation system can operate in accordance with one or more embodiments.

FIG. 14 illustrates an example series of acts for generating a SAFE molecular representation in accordance with one or more embodiments.

FIG. 15 illustrates an example series for training a large language model to generate a sequential attachment-based fragment embedding (SAFE) molecular string representation in accordance with one or more embodiments.

FIG. 16 illustrates a block diagram of an example computing device for implementing one or more embodiments of the present disclosure.

### DETAILED DESCRIPTION

This disclosure describes one or more embodiments of a digital molecular structure generation system that generates sequential attachment-based fragment embedding (SAFE) molecular string representations representing molecules as order agnostic sequences of interconnected fragment blocks for processing via large language models for downstream molecular design tasks. In one or more implementations, the digital molecular structure generation system converts molecular string representations (e.g., SMILES molecular string representations) into SAFE molecular string representations that include fragments with separation character connections between the fragments and ring link characters to simulate fragment links. In addition, the digital molecular structure generation system can also utilize the SAFE molecular string representations (having order agnostic sequences of interconnected fragment blocks to represent molecular compounds) to train a large language model to generate (or complete) additional SAFE molecular string representations for a variety of fragment-based molecular design tasks (e.g., de novo molecular compound generation tasks, scaffold decoration and motif extension tasks, linker design and scaffold morphing tasks, and/or molecular superstructure generation tasks).

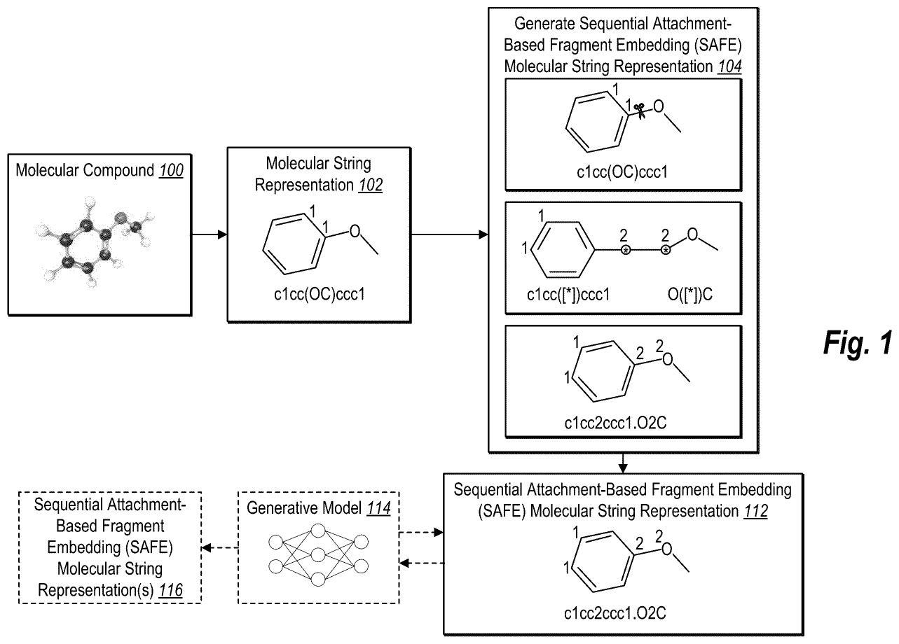

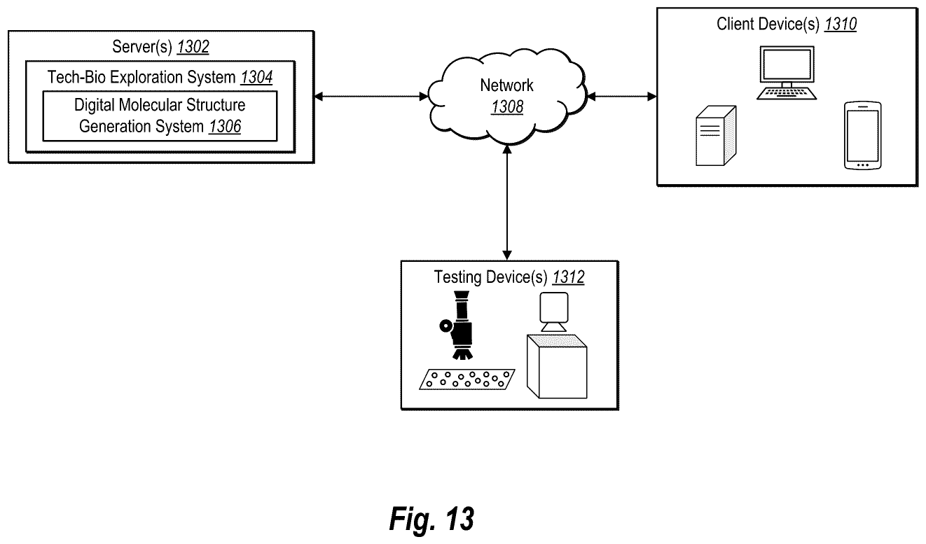

For example, FIG. 1 illustrates an overview of a digital molecular structure generation system 1306 (e.g., as described in FIG. 13) generating a SAFE molecular string representations representing molecules as order agnostic sequences of interconnected fragment blocks. In addition, FIG. 1 also illustrates an overview of the digital molecular structure generation system 1306 utilizing a SAFE molecular string representation to train a generative model for a variety of fragment-based molecular design tasks.

For instance, as shown in FIG. 1, the digital molecular structure generation system 1306 identifies a molecular string representation 102 (e.g., “c1cc(OC)ccc1”) that indicates virtual connections between atom representations of a molecular compound 100 (e.g., through ring structure identifiers). Indeed, the molecular string representation 102 represents a SMILES molecular string representation. Furthermore, as shown in an act 104 of FIG. 1, the digital molecular structure generation system 1306 generates a sequential attachment-based fragment embedding (SAFE) molecular string representation from the molecular string representation 102.

As illustrated in the act 104, to generate a SAFE molecular string representation 112, the digital molecular structure generation system 1306 can extract fragments from the molecular string representation 102 to generate a set of fragments (e.g., “c1cc([*])ccc1” and “O([*])C”). In some cases, the digital molecular structure generation system 1306 utilizes a bond slicing algorithm to break bonds in a molecular string representation (e.g., the molecular string representation 102) to generate the set of fragments (e.g., “c1cc([*])ccc1” and “O([*])C”). As further shown in FIG. 1, the digital molecular structure generation system 1306 also extracts (and preserves) attachment point indicators to indicate fragment links in the molecular string representation 102 (e.g., the attachment point indicators “[*]”). In particular, the digital molecular structure generation system 1306 can utilize (or add) the attachment point indicators to represent ring closing bonds that connect two or more fragments.

As further illustrated in the act 104, to generate the SAFE molecular string representation 112, the digital molecular structure generation system 1306 concatenates the fragments from the set of fragments (e.g., “c1cc([*])ccc1” and “O([*])C”) utilizing separation characters (e.g., “.”) between the fragments to generate a linked fragment string. Indeed, the digital molecular structure generation system 1306 can generate the linked fragment string (e.g., “c1cc([*])ccc1.O([*])C”) by concatenating the fragments using a separation character. Moreover, as shown in the act 104, the digital molecular structure generation system 1306 also generates ring link characters in the linked fragment string to represent the attachment points for specific fragment links (e.g., to accurately indicate bonds between different fragments). For instance, as shown in the act 104 of FIG. 1, the digital molecular structure generation system 1306 generates a ring link character (“2”) in the linked fragment string in place of the attachment point indicator (“[*]”) to represent fragment links. In some cases, the digital molecular structure generation system 1306 utilizes the ring link characters to indicate distinctive fragment links between fragments identified in the molecular string representation 102.

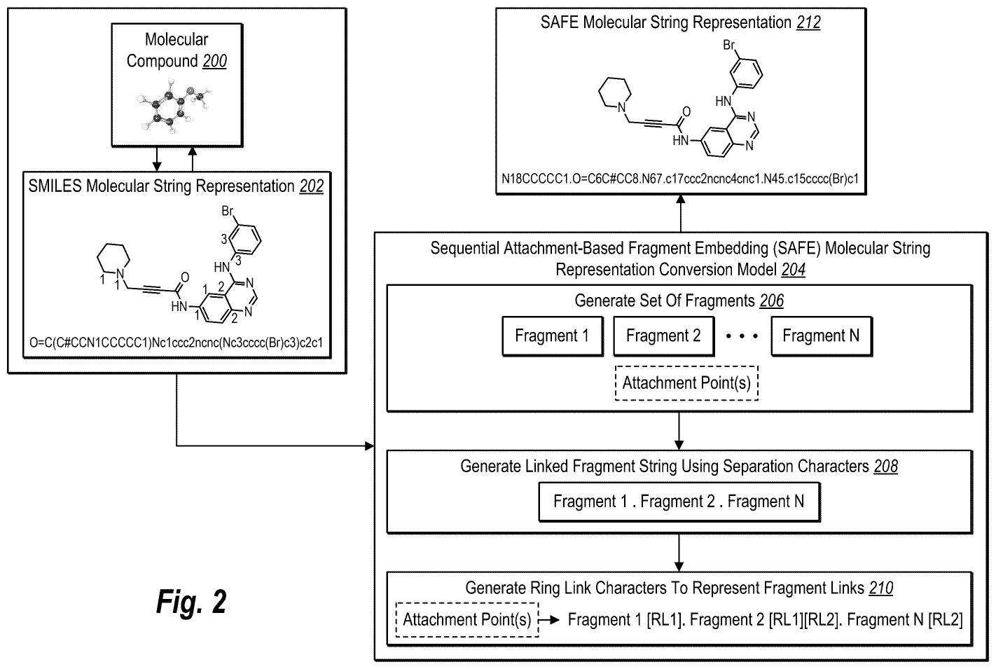

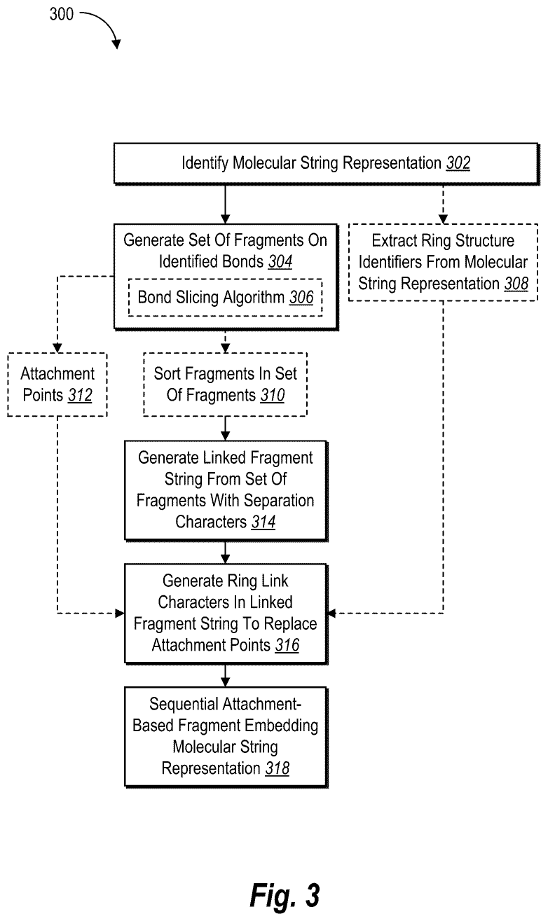

Utilizing the separation character and ring link character in the act 104, the digital molecular structure generation system 1306 generates the SAFE molecular string representation 112 (e.g., “c1cc2ccc1.O2C”) which represents the molecular string representation 102 as an order agnostic sequence of interconnected fragment blocks. Indeed, the digital molecular structure generation system 1306 can generate the SAFE molecular string representation 112 agnostic of fragment block order (e.g., “c1cc2ccc1.O2C” or “O2C.c1cc2ccc1”) while representing the same molecular string representation 102. The digital molecular structure generation system 1306 generating a SAFE molecular string representation is described in greater detail below (e.g., in reference to FIGS. 2 and 3).

Although one or more embodiments described herein illustrate the digital molecular structure generation system 1306 generating a SAFE molecular string representation from a SMILES molecular string representation, the digital molecular structure generation system 1306 can generate a SAFE molecular string representation from a variety of molecular string representation notations in accordance with one or more implementations herein. Furthermore, the digital molecular structure generation system 1306 can generate a SAFE molecular string representation from a molecular string representation having a variety of fragments and/or a variety of bonds per fragments.

As used herein, the term “molecular compound” (sometimes referred to as “compound” or “molecule compound”) refers to a chemical compound having atoms with bonds to form a stable molecule (e.g., a drug or medicine). Indeed, in one or more instances, a molecular compound includes a substance composed of molecules designed to interact with specific biological targets (e.g., proteins, enzymes, or receptors).

Furthermore, as used herein, the term “molecular representation” (sometimes referred to as “molecular string representation”) refers to a notation that depicts a structure, composition, and/or function of a molecular compound. For instance, a molecular representation can include, but is not limited to, a molecular formula, a structural formula, or a chemical notation. In one or more instances, a molecular representation can include a chemical notation that represents molecular structures (e.g., ring structures, attachment points) in a text (or string) format for utilization in computational models. As an example, a molecular string representation can include a Simplified Molecular Input Line Entry System (SMILES), an International Chemical Identifier (InChl), and/or a Group Self-Referencing Embedded Strings (Group SELFIES).

Additionally, as used herein, the term “ring structure” refers to a molecular structure in which one or more atoms are connected in a closed loop. In particular, a ring structure can include an opening ring and a closing ring. Furthermore, as used herein, the term “attachment point” refers to a location within a molecule (or a molecule representation) (e.g., a ring structure) where an atom or group of atoms are attached (or connected).

As used herein, the term “sequential attachment-based fragment embedding molecular representation” (sometimes referred to as “SAFE representation” or “SAFE string representation”) refers to a molecular representation that indicates linked fragment blocks in a string with separation characters and ring link characters. Indeed, a SAFE representation depicts linked fragment blocks in a string of order agnostic fragment blocks designated with separation characters to specify individual fragments and ring link characters to specify fragment links with other fragment blocks in a molecular compound. In particular, a SAFE string representation includes a molecular representation generated in accordance with one or more implementations herein.

As used herein, the term “fragment” refers to a portion or piece of a molecule that represents an independent functional group (e.g., one or more atoms) with an identify and property within a molecule. For instance, a molecular compound can include a set of fragments connected to form a structure. As an example, a fragment can include, but is not limited to, a benzine ring or other pharmaceutical compound, an amine, a ketone, an amino acid (protein), and/or a synthetic compound.

As used herein, the term “separation character” refers to a string character or symbol that indicates a marker within a SAFE string representation to differentiate between fragments of a molecular compound. For instance, a separation character can include a string character or symbol that depicts a partition between two fragments in a molecular compound. A SAFE string representation can include multiple separation characters between multiple fragments. As an example, a separation character can include a “.” character, a “|” character, and/or a “-” character between fragments.

As used herein, the term “ring link character” refers to a string character or symbol that indicates an attachment point or fragment link within a SAFE string representation. In particular, a ring link character can specify a particular linkage between two or more fragments. For example, a ring link character can include a specific linking character that designates a specific link between fragments. In some cases, the ring link character includes one or more digits (e.g., a ring link digit) that each represent different fragment links in a SAFE string representation. In one or more instances, a ring link character can include a variety of characters or symbols, such as, alphanumerical characters, symbols, numerical characters.

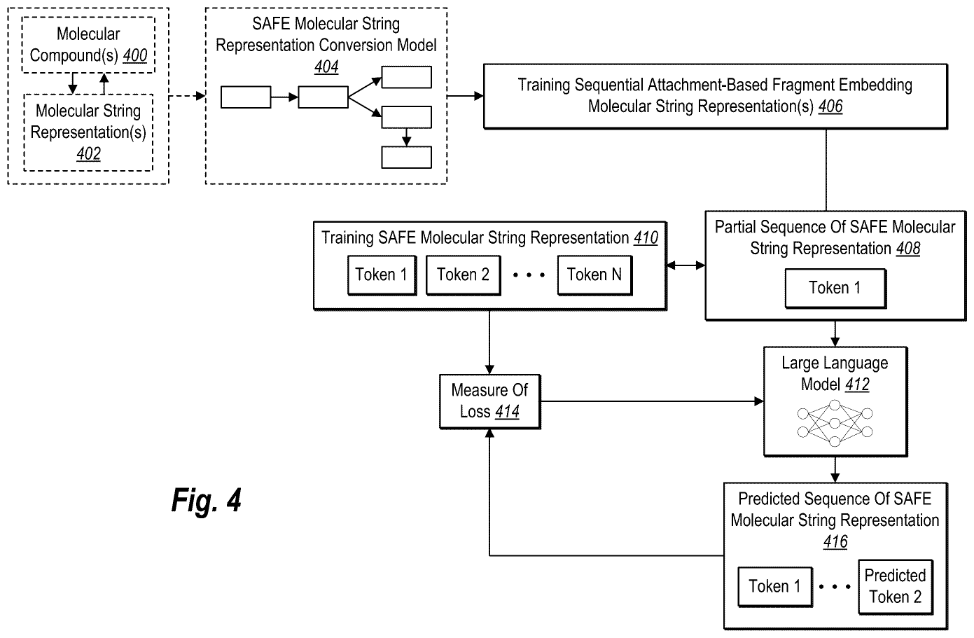

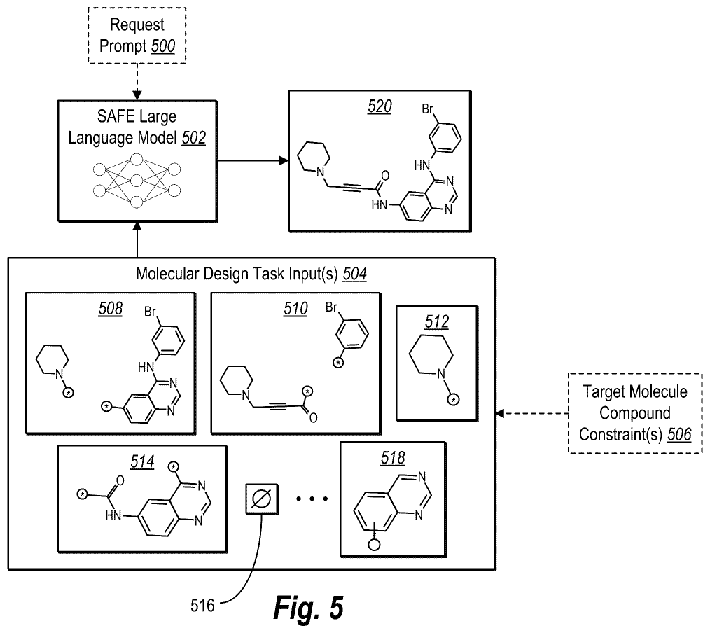

As further shown in FIG. 1, the digital molecular structure generation system 1306 utilizes the SAFE molecular string representation 112 with a generative model 114 (e.g., a large language model). For instance, the digital molecular structure generation system 1306 can train the generative model 114 for a variety of fragment-based molecular design tasks. Indeed, as shown in FIG. 1, the digital molecular structure generation system 1306 can utilize the SAFE molecular string representation 112 to train the generative model 114 to generate one or more SAFE molecular string representation(s) 116 that accomplish a variety of downstream fragment-based molecular design tasks (e.g., de novo molecular compound generation tasks, scaffold decoration and motif extension tasks, linker design and scaffold morphing tasks, and/or molecular superstructure generation tasks). The digital molecular structure generation system 1306 utilizing SAFE molecular string representation(s) with generative models (e.g., large language models) is described in greater detail below (e.g., in reference to FIGS. 4 and 5).

As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more (deep) learning techniques (e.g., supervised or unsupervised learning) to improve in accuracy and/or effectiveness. Example machine learning models include various types of decision trees, support vector machines, Bayesian networks, random forest models, or neural networks (e.g., deep neural networks, generative adversarial neural networks, convolutional neural networks, recurrent neural networks, large language models, or diffusion neural networks). Similarly, the term “machine learning data” refers to information, data, or files generated or utilized by a machine learning model. Machine learning data can include training data, machine learning parameters, or embeddings/predictions generated by a machine learning model.

As used herein, the term “language machine learning model” refers to a machine learning model that analyzes a language input (e.g., text or verbal input) to generate a predicted output. For instance, a language machine learning model includes a neural network that generates text based on an input text or query. The digital molecular structure generation system 1306 can utilize a variety of architectures for a language machine learning model, such as a large language model or other transformer neural network model.

For instance, a large language model includes one or more neural networks capable of processing natural language text to generate outputs that range from predictive outputs, analyses, one or more SAFE molecular string representations, or combinations of data within stored content items. In particular, a large language model can include parameters trained (e.g., via deep learning) on large data volumes to learn patterns and rules of language for summarizing and/or generating digital content. Examples of large language model include BLOOM, Bard AI, ChatGPT (e.g., GPT-3, GPT-4, etc.), LaMDA, and/or DialoGPT. Moreover, in some embodiments a language transformer model includes bidirectional encoder representations (BERT), Robustly optimized BERT (RoBERTa), and other text transformer models. Indeed, the digital molecular structure generation system 1306 can utilize a large language model trained to learn patterns and rules defined by molecular compound structures to generate SAFE molecular representations and/or to perform various downstream fragment-based molecular design tasks.

As used herein, the term “prompt” refers to a set of input instructions to a large language model (or other machine learning model) to cause the large language model to generate a particular output (or perform a particular task). Indeed, a prompt can include an input string of text that includes request for a large language model (e.g., generate a novel molecular compound, generate a molecular compound that includes properties for Central Nervous System (CNS) penetration). In one or more cases, a prompt can include a text input and/or a voice command.

As mentioned above, although existing systems can utilize molecular string representations, such as SMILES, these conventional systems often have a number of technical shortcomings with regard to flexibility and accuracy. For instance, many conventional systems cannot easily or accurately utilize SMILES molecular string representations for AI-driven molecular design tasks and computational drug discovery. In particular, AI-driven molecular design tasks and computational drug discovery often demand the preservation certain scaffolds or core chemical substructures (which serve as a backbone for molecular design processes). Indeed, preserving these groups and constraints often stems from their crucial role in a molecule's biological activity. In many instances, conventional systems are unable to incorporate such constraints when relying on SMILES molecular string representations (or many other conventional molecular string representations).

To illustrate, in many conventional systems, a SMILES molecular string representation is unable to provide a contiguous representation of molecular substructures. This limitation often hinders tasks, such as adding structures to a molecule's scaffold and connecting fragments. Such limitations also often limit SMILES representations' usefulness in improving potential drug candidates (e.g., during lead optimization efforts, during AI-driven molecular design tasks). Indeed, in many conventional systems, SMILES molecular string representations lack robustness to minor changes and struggle with ensuring validity and integrity of fragments in deep learning-based molecular design. In addition, the SMILES molecular string representations also often underperform in molecular search and substructure matching tasks.

Many other approaches (e.g., Self-Referencing Embedded Strings (SELFIES)), Group SELFIES) aim to resolve the deficiencies of SMILES molecular string representations but also have a number of technical shortcomings with regard to flexibility and accuracy. For instance, SELFIES and Group SELFIES improve on the robustness and validity issues via deep generative modeling through a recursive approach, however such representations lack simplicity, are difficult to interpret, and are not compact. Furthermore, these approaches often also fail to consistently uphold the integrity of scaffolds and fragments for several molecular generation tasks. In addition, such approaches fail to facilitate deep generative fragment-based molecule design without extensive, task-specific engineering of training processes and molecule generation steps, bespoke model architectures, or goal-directed optimization frameworks.

In some instances, many conventional systems utilize graph-based methods to create molecular representations that facilitate AI-driven molecular design tasks. However, many graph-based methods encounter difficulties when extending design tasks to scaffold-based generation, linker-design, and generating molecules with unseen building blocks. Indeed, many of these approaches experience difficulties in creating novel cyclic structures not seen during training. Furthermore, some conventional systems utilize graph-based models that are trained on the SMILES molecular string representations, however these models often fail to guarantee validity of generated molecules and the presence of input scaffold constraints. In particular, many conventional systems are unable to (e.g., due to incapability or due to additional required engineering) facilitate one or more molecular design tasks, such as de novo molecular compound generation tasks, scaffold decoration and motif extension tasks, linker design and scaffold morphing tasks, and/or molecular superstructure generation tasks.

As suggested by the foregoing, the digital molecular structure generation system 1306 provides a variety of technical advantages relative to conventional systems. Indeed, the digital molecular structure generation system 1306 generates and utilizes sequential attachment-based fragment embedding (SAFE) molecular string representations that represent molecules as order agnostic sequences of interconnected fragment blocks that can flexibly and accurately be utilized with large language models for downstream molecular design tasks. Indeed, the digital molecular structure generation system 1306 can generate SAFE molecular string representations by converting molecular string representations (e.g., SMILES molecular string representations) as an order agnostic sequence of interconnected fragment blocks while maintaining compatibility with existing molecular string representation parsers (e.g., SMILES parsers).

By being order agnostic sequences, the SAFE molecular string representations enable the digital molecular structure generation system 1306 to flexibly and accurately utilize the SAFE molecular string representations with generative models for one or more molecular design tasks. Indeed, the SAFE molecular string representations preserve the integrity of molecular scaffolds and fragments. Additionally, the digital molecular structure generation system 1306 can easily utilize SAFE molecular string representations as simple sequence completion problems that enable accuracy and flexibility in molecular design tasks, such as de novo molecular compound generation tasks, scaffold decoration and motif extension tasks, linker design and scaffold morphing tasks, and molecular superstructure generation tasks. Moreover, the SAFE molecular string representations (generated by the digital molecular structure generation system 1306) also facilitate autoregressive generation which flexibly bypasses the necessity for intricate decoding schemes or graph-based models (in molecular design generative tasks).

Additionally, the digital molecular structure generation system 1306 generates SAFE molecular string representations as a collection of connected fragments that remain valid as other molecular string representations (e.g., a SMILES representation). Accordingly, while enabling many AI-driven molecular design tasks (which is not often possible in conventional systems), the SAFE molecular string representations generated by the digital molecular structure generation system 1306 remain compatible with other molecular string representations (e.g., a SMILES representation), such that the SAFE molecular string representations are backward compatible with many existing molecular string representation parsers (e.g., SMILES parsers). For instance, the underlying molecular graph remains unaffected by the arrangement of fragments within a SAFE molecular string representation to ensure that data augmentation techniques for generative models (corresponding to other molecular string representations), such as randomization, remain applicable to the SAFE molecular string representation.

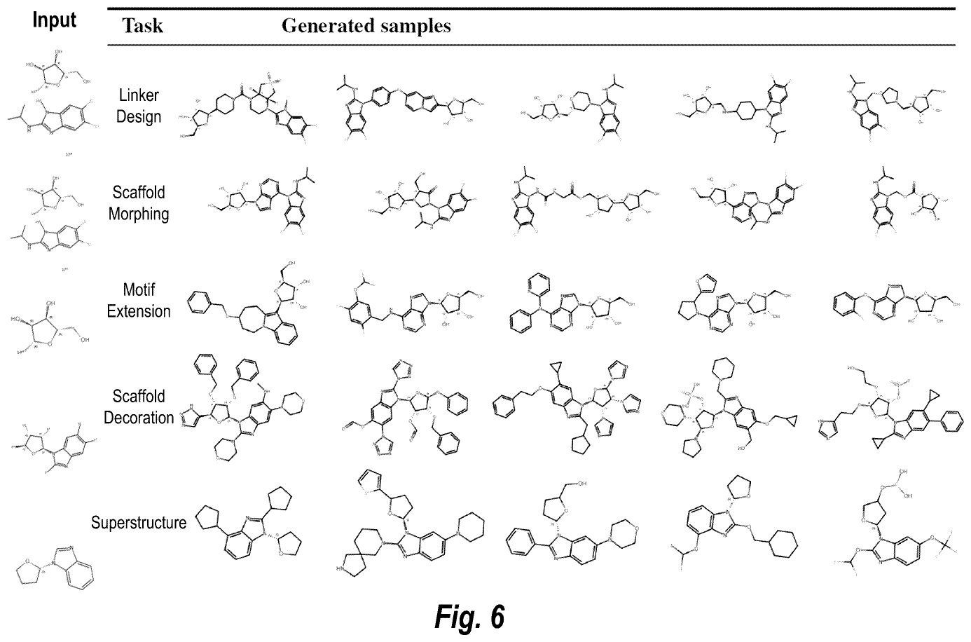

Indeed, experimental results illustrated in FIGS. 6-12 demonstrate a variety of technical advantages provided by one or more implementations of the digital molecular structure generation system 1306.

As mentioned above, the digital molecular structure generation system 1306 can generate sequential attachment-based fragment embedding (SAFE) molecular string representations from other molecular string representations (e.g., SMILES molecular string representations). For example, FIG. 2 illustrates the digital molecular structure generation system 1306 generating a SAFE molecular string representation. In particular, FIG. 2 illustrates the digital molecular structure generation system 1306 converting a SMILES molecular string representation to a SAFE molecular string representation.

As shown in FIG. 2, the digital molecular structure generation system 1306 identifies a SMILES molecular string representation 202 for a molecular compound 200. Moreover, as shown in FIG. 2, the digital molecular structure generation system 1306 utilizes a SAFE molecular string representation conversion model 204 to convert the SMILES molecular string representation 202 (e.g., “O=C(#CCN1CCCCC1)Nc1ccc2ncnc(Nc3cccc(Br)c3)c2c1”) into a SAFE molecular string representation 212 (e.g., “N18CCCCC1.O=C6C#CC8.N67.c17ccc2ncnc4cnc1.N45.c15cccc(Br)c1”).

To convert the SMILES molecular string representation 202 into the SAFE molecular string representation 212, as shown in act 206 of FIG. 2, the digital molecular structure generation system 1306, via the SAFE molecular string representation conversion model 204, generates a set of fragments from the SMILES molecular string representation 202. As an example, the digital molecular structure generation system 1306 generates a set of fragments (e.g., fragment 1, fragment 2, fragment N) to represent one or more fragments in the SMILES molecular string representation 202. In addition, as shown in the act 206, the digital molecular structure generation system 1306 can also determine attachment point(s) for the set of fragments to indicate relationships or fragment links between the extracted fragments (as indicated by the SMILES molecular string representation 202).

Furthermore, as shown in an act 208 of FIG. 2, the digital molecular structure generation system 1306, via the SAFE molecular string representation conversion model 204, generates a linked fragment string from the set of fragments using separation characters. For example, as shown in FIG. 2, the digital molecular structure generation system 1306 can concatenate the set of fragments (from the act 206) with separation characters (“.”) to generate the linked fragment string (e.g., “Fragment1.Fragment2.FragmentN”). Although one or more embodiments illustrates the digital molecular structure generation system 1306 utilizing a “.” character as a separation character, the digital molecular structure generation system 1306 can utilize a variety of separation characters (e.g., “;”, “|”, “_”).

In addition, as shown in an act 210 of FIG. 2, the digital molecular structure generation system 1306, via the SAFE molecular string representation conversion model 204, generates ring link characters to represent fragment links in the linked fragment string (of the act 208). In particular, as shown in the act 210 of FIG. 2, the digital molecular structure generation system 1306 utilizes attachment point(s) extracted (or identified) from the SMILES molecular string representation 202 that indicate connections between the fragments to add (or generate) ring link characters for the fragments that indicate attachment point locations between the fragments. For instance, as shown in the act 210, the digital molecular structure generation system 1306 utilizes (or generates) a ring link character “RL1” for “Fragment 1” and for “Fragment 2” to indicate a fragment link between “Fragment 1” and “Fragment 2.” As also shown in the act 210, the digital molecular structure generation system 1306 utilizes (or generates) a ring link character “RL2” for “Fragment 2” and for “Fragment N” to indicate a fragment link between “Fragment 2” and “Fragment N.” Indeed, the digital molecular structure generation system 1306 can generate multiple ring link characters for fragments to indicate multiple fragment links between fragments (e.g., a fragment can be represented with two or more ring link characters to illustrate a fragment link with more than one fragment).

In some cases, the digital molecular structure generation system 1306 utilizes ring link digits (e.g., 1, 2, 3, 4, 5) as the ring link characters. For instance, in the act 206, the digital molecular structure generation system 1306 can generate a ring link character (or digit) of “1” for “RL1” and a ring link character (or digit) of “2” for “RL2.” Although one or more embodiments described herein utilize specific ring link characters (or digits), the digital molecular structure generation system 1306 can utilize a variety of ring link characters (e.g., alphanumerical characters, symbols, numerical characters).

As shown in FIG. 2, the digital molecular structure generation system 1306, upon generating the linked fragment string and generating ring link characters for the linked fragment string, generates the SAFE molecular string representation 212. For instance, as shown in FIG. 2, the SAFE molecular string representation 212 (e.g., “N18CCCCC1.O=C6C#CC8.N67.c17ccc2ncnc4cnc1.N45.c15cccc(Br)c1”) includes fragment blocks represented by fragment strings, separation characters, and ring link characters.

Furthermore, in one or more instances, the digital molecular structure generation system 1306 can generate a SAFE molecular string representation (or linked fragment string) utilizing a varying order of the fragments. For instance, the digital molecular structure generation system 1306 can generate, in the act 208, a linked fragment string by concatenating the set of fragments as, but not limited to, “Fragment1.FragmentN.Fragment2” or “Fragment2.Fragment1.FragmentN”) and also generate corresponding ring link characters in the act 210. As an example, the digital molecular structure generation system 1306 can generate the SAFE molecular string representation 212 utilizing varying permutations, such as “N18CCCCC1.O=C6C#CC8.N67.c17ccc2ncnc4cnc1.c15cccc(Br)c1.N45”. Indeed, the digital molecular structure generation system 1306 generates and utilizes a SAFE molecular string representation that is permutable while preserving the same fragment link connections (because the ring link characters continue to specify fragment links in different arrangements of the fragment blocks).

Furthermore, FIG. 3 illustrates a flow diagram 300 of the digital molecular structure generation system 1306 generating a SAFE molecular string representation. As shown in act 302 of FIG. 3, the digital molecular structure generation system 1306 identifies a molecular string representation. Moreover, as shown in an act 304 of FIG. 3, the digital molecular structure generation system 1306 generates a set of fragments on identified bonds from the molecular string representation. In some cases, as shown in an act 306 of FIG. 3, the digital molecular structure generation system 1306 utilizes a bond slicing algorithm to generate the set of fragments from the molecular string representation.

In one or more instances, the digital molecular structure generation system 1306 utilizes a bond slicing algorithm to determine fragments on a desired set of bonds from ring structures represented in a molecular string representation (e.g., a SMILES molecular string representation). For instance, in one or more implementations, the digital molecular structure generation system 1306 utilizes a breaking of retrosynthetically interesting chemical substructures (BRICS) algorithm as the bond slicing algorithm as described in Degen et al., On the Art of Compiling and Using ‘Drug-like’ Chemical Fragment Spaces, ChemMedChem: Chemistry Enabling Drug Discovery, 3(10):1503-1507 (2008) (hereinafter “Degen”), which is incorporated herein by reference in its entirety. Although one or more implementations of the digital molecular structure generation system 1306 utilizes a BRICS algorithm, the digital molecular structure generation system 1306 can utilize various bond slicing algorithms, such as, but not limited to, match molecular pair method as described in Hussain et al., Computationally Efficient Algorithm to Identify Matched Molecular Pairs (MMPS) in Large Data Sets, Journal of Chemical Information and Modeling, 50(3):339-348 (2010) (hereinafter “Hussain”), RECAP as described in Lewell et al., Recap Retrosynthetic Combinatorial Analysis Procedure: A Powerful New Technique for Identifying Privileged Molecular Fragments with Useful Applications in Combinatorial Chemistry, Journal of Chemical Information and Computer Sciences, 38(3):511-522 (1998) (hereinafter “Lewell”), and/or custom patterns, each of which are incorporated herein by reference in their entirety.

Furthermore, as shown in an act 310 of FIG. 3, the digital molecular structure generation system 1306 can sort fragments in the set of fragments. In particular, the digital molecular structure generation system 1306 can sort the fragments in the set of fragments utilizing characteristics of the fragments. For instance, the digital molecular structure generation system 1306 can sort the fragments based on characteristics, such as, but not limited to size (e.g., size in descending order, size in ascending order), corresponding ring structures, and/or attachment point indicators.

Additionally, as shown in FIG. 3, the digital molecular structure generation system 1306 can identify attachment points 312 (e.g., attachment point indicators) for the fragments from the molecular string representation. In one or more instances, the digital molecular structure generation system 1306 can extract attachment point indicators from a molecular representations string by identifying from a sequential of a molecular string representation (e.g., a SMILES string) a connection between one or more fragments. In some cases, the digital molecular structure generation system 1306 indicates (or flags) attachment point indicators with fragments within the set of fragments. In one or more instances, the digital molecular structure generation system 1306 utilizes a mapping reference (e.g., a table, a tree structure, an array) to map attachment points between fragments.

Moreover, as shown in an act 308 of FIG. 3, the digital molecular structure generation system 1306 can also extract ring structure identifiers from a molecular string representation. For instance, the digital molecular structure generation system 1306 can identify, from a SMILES string, ring structures marked using digits to identify opening and/or closing ring atom(s) denoting a virtual connection between the corresponding atoms. As an example, the digital molecular structure generation system 1306 can extract ring structure identifiers “1,” “2,” and “3” from a SMILES string of “O=C(#CCN1CCCCC1)Nc1ccc2ncnc(Nc3cccc(Br)c3)c2c1”.

Furthermore, as shown in an act 314, the digital molecular structure generation system 1306 generates a linked fragment string from the set of fragments with separation characters (in accordance with one or more implementations herein). Furthermore, as shown in act 316 of FIG. 3, the digital molecular structure generation system 1306 generates ring link characters in the linked fragments string to replace attachment points of the fragments with ring link characters that indicate particular fragment links in the molecular string representation. In particular, the digital molecular structure generation system 1306 can utilize the attachment points 312 (and ring structure identifiers) to generate ring link characters in the linked fragment string. Indeed, in the act 316, the digital molecular structure generation system 1306 generates ring link characters in the linked fragments string (in accordance with one or more implementations herein) to generate a sequential attachment-based fragment embedding molecular string representation 318.

In one or more instances, the digital molecular structure generation system 1306 can generate a SAFE molecular string representation in accordance with the following Algorithm 1:

```
Algorithm 1
procedure ToSAFE(molecule)
      ring_{digits} ← extract all unique ring digits from molecule
      fragments ← fragment molecule on specified bonds [symbol]   using a bond slicing algorithm
      Sort fragments in fragments by size (in descending order)
      fragments_str ← { }
      for each frag in fragments do
        Add smiles of frag to fragments_str
      safe_str ← join all elements in fragments_str with “.”
      attach_pos ← extract all attachment points from safe_str
      i ← max(ring_digits) + 1 [symbol]   Find the next possible ring digits
      for each attach in attach_pos do
        Replace attach in safe_str with i
        Increment i by 1
      return safe_str
```

To illustrate, in the above-mentioned Algorithm 1, the digital molecular structure generation system 1306 can extract unique ring identifiers from a molecule and fragment the molecule on a desired set of bonds (e.g., using a bond slicing algorithm). Indeed, the fragment substructures can represent synthetically accessible building blocks that are present in drug-like compounds. Moreover, the digital molecular structure generation system 1306 can sort the extracted fragments by size. Furthermore, the digital molecular structure generation system 1306 can concatenate the fragments using a separation character “.” to mark new fragments in the representation (while preserving their corresponding attachment points). To construct the SAFE string representation, the digital molecular structure generation system 1306 can iterate over the numbered attachment points and replace them with a ring link character (e.g., i) to simulate fragment linking. The ring link characters create virtual connections between fragments resulting in a set of linked fragments (indicated by the separation character).

Furthermore, in some cases, the digital molecular structure generation system 1306 can canonicalize SAFE string representations such that multiple valid forms of a molecular representation yield a unique representation by enforcing a decoding order on SMILES characters within fragment and on fragment orders within the converted SAFE string representation.

Additionally, as mentioned above, the digital molecular structure generation system 1306 can utilize SAFE molecular string representations to enable various downstream fragment-based molecular design tasks via large language models. For instance, FIG. 4 illustrates the digital molecular structure generation system 1306 utilizing SAFE molecular string representations to train a generative model (e.g., a large language model). Indeed, FIG. 4 illustrates the digital molecular structure generation system 1306 training the generative model (e.g., a large language model) using SAFE molecular string representations to generate SAFE molecular string representations for downstream fragment-based molecular design tasks (e.g., de novo molecular compound generation tasks, scaffold decoration and motif extension tasks, linker design and scaffold morphing tasks, molecular superstructure generation tasks).

As shown in FIG. 4, the digital molecular structure generation system 1306 identifies training sequential attachment-based fragment embedding (SAFE) molecular string representation(s) 406. In some cases, the digital molecular structure generation system 1306 identifies the training SAFE string representation(s) from a repository of training data (e.g., on a repository or data storage of a tech-bio exploration system 1304 (as described in FIG. 13). In some instances, as shown in FIG. 4, the digital molecular structure generation system 1306 can generate the training SAFE string representation(s). For instance, as shown in FIG. 4, the digital molecular structure generation system 1306 can identify molecular compound(s) 400 with corresponding molecular string representation(s) 402 (e.g., SMILES strings). Subsequently, as shown in FIG. 4, the digital molecular structure generation system 1306 can utilize the molecular string representation(s) 402 with a SAFE molecular string representation conversion model 404 to generate the training SAFE string representation(s) 406 (in accordance with one or more implementations herein).

Additionally, in some cases, the digital molecular structure generation system 1306 can utilize permutations of SAFE molecular string representations as the training SAFE string representation(s). For instance, the digital molecular structure generation system 1306 can generate randomized training SAFE string representation(s) by generating random permutations of SAFE molecular string representations (e.g., by randomizing fragment locations within the string representation). For example, the digital molecular structure generation system 1306 can generate varying permutations of SAFE string representation(s) as described above (e.g., in reference to FIG. 2).

Furthermore, as shown in FIG. 4, the digital molecular structure generation system 1306 utilizes the training SAFE string representation(s) 406 to generate a partial sequence of SAFE molecular string representation 408. Moreover, as illustrated in FIG. 4, the digital molecular structure generation system 1306 utilizes the partial sequence of SAFE molecular string representation 408 with a large language model 412 to predict a predicted sequence of the SAFE molecular string representation 416 (e.g., a completion task). As shown in FIG. 4, the digital molecular structure generation system 1306 can tokenize the training SAFE string representation(s) 406 and input, as a tokenized partial sequence of SAFE molecular string representation 408, a subset of tokens (e.g., “token 1”) from tokens (e.g., “token 1,” “token 2,” . . . “token N”) corresponding to a training SAFE string representation. In some cases, the digital molecular structure generation system 1306 can, to generate the partial sequence of SAFE molecular string representation 408, mask one or more tokens (e.g., the subset of tokens) from tokens corresponding to the training SAFE string representation.

Indeed, as further illustrated in FIG. 4, the digital molecular structure generation system 1306 utilizes the large language model 412 to generate a predicted token as the predicted sequence of the SAFE molecular string representation 416 (e.g., “predicted token 2”). For example, the predicted token can represent a portion (or subsequent portion) of a predicted SAFE molecular string representation (e.g., completion of an incomplete or masked SAFE molecular string representation). For instance, the digital molecular structure generation system 1306 can utilize the large language model 412 to generate a predicted subsequent token (as the predicted token) for the partial sequence of SAFE molecular string representation 408. In some cases, the digital molecular structure generation system 1306 can utilize the large language model 412 to generate a predicted completion of a SAFE molecular string representation (as the predicted token) for masked training SAFE molecular string representation (e.g., the partial sequence of SAFE molecular string representation 408).

As further shown in FIG. 4, the digital molecular structure generation system 1306 utilizes the predicted sequence of the SAFE molecular string representation 416 with a training SAFE molecular string representation 410 to generate a measure of loss 414. In particular, the digital molecular structure generation system 1306 can compare the predicted sequence of the SAFE molecular string representation 416 to the ground truth training SAFE molecular string representation 410 to determine an accuracy of the predicted sequence of the SAFE molecular string representation 416 (e.g., if “token 2” matches “predicted token 2”).

Indeed, the digital molecular structure generation system 1306 can utilize the comparison to generate the measure of loss 414 to quantify errors (or inaccuracies) between the predicted sequence of the SAFE molecular string representation 416 and the original (ground truth) training SAFE molecular string representation 410. Moreover, the digital molecular structure generation system 1306 utilizes the measure of loss 414 with the large language model 412 to adjust (or modify) parameters of the large language model 412 (e.g., via back propagation). In one or more instances, the digital molecular structure generation system 1306 iteratively repeats the determination and utilization of measures of losses as shown in FIG. 4 to train the large language model 412. For instance, the digital molecular structure generation system 1306 utilizes the measure of loss 414 to modify parameters of the large language model 412 to accurately generate SAFE molecular string representations (e.g., by adjusting parameters to output predicted SAFE tokens that reduce or minimize the measure of loss 414).

In some cases, the digital molecular structure generation system 1306 utilizes a dataset to generate training SAFE string representation(s). For instance, the digital molecular structure generation system 1306 can generate (or identify) SMILES strings from a dataset of molecules. As an example, the digital molecular structure generation system 1306 can utilize a dataset of molecules, such as the ZINC library as described in Irwin et al., ZINC—A Free Database of Commercially Available Compounds for Virtual Screening, Journal of Chemical Information and Modeling, 45(1):177-182 (2005) (hereinafter “Irwin”) and the UniChem library as described in Chambers, et al., UniChem: A Unified Chemical Structure Cross-Referencing and Identifier Tracking System, Journal of Cheminformatics, 5(1):3 (2013) (hereinafter “Chambers”), each of which are incorporated herein by reference in their entirety. Moreover, the digital molecular structure generation system 1306 can convert the SMILES strings from the dataset of molecules (described above) into SAFE molecular string representations in accordance with one or more implementations herein.

In addition, the digital molecular structure generation system 1306 can generate tokens (or fragments) for the training SAFE string representation(s) to utilize in a generative model (e.g., a large language model). For instance, in some cases, the digital molecular structure generation system 1306 can identify expressions (e.g., common regular expressions) for SMILES representations as described in Schwaller et al., Molecular Transformer: A Model for Uncertainty-Calibrated Chemical Reaction Prediction, ACS Central Science, 5(9):1572-1583 (2019), which is incorporated herein by reference in its entirety. Indeed, the digital molecular structure generation system 1306 can utilize the expressions to generate a vocabulary of tokens represented within a dataset of SMILES representations (or SAFE representations).

Furthermore, the digital molecular structure generation system 1306 can utilize a tokenizer to generate tokens that represent various expressions for molecular representation syntax (e.g., SMILES syntax and/or SAFE syntax). To illustrate, the digital molecular structure generation system 1306 can utilize a tokenizer to generate tokens to represent the above-described expressions or vocabulary from molecular representation syntax. Indeed, in some cases, the digital molecular structure generation system 1306 utilizes a variety of tokenizers, such as, a byte-pair encoding (BPE) tokenizer, wordpiece tokenization approaches, and/or sentence piece tokenization approaches.

In some instances, the digital molecular structure generation system 1306 also generates one or more special tokens. In particular, the digital molecular structure generation system 1306 can generate tokens to represent an end-of-sequence (e.g., EOS) to indicate the end of a SMILES or SAFE representation, a beginning-of-sequence (e.g., BOS) to indicate the beginning of a SMILES or SAFE representation, a mask token (e.g., MASK) to represent a masked token. Indeed, the digital molecular structure generation system 1306 can generate a variety of special tokens (e.g., EOS, BOS, UNK, MASK, PAD).

In one or more implementations, the digital molecular structure generation system 1306 utilizes the generative model (e.g., the large language model) to generate (or learn) a token distribution for a predicted token of a (partial) SAFE molecular string representation. In particular, the digital molecular structure generation system 1306 utilizes the generative model to generate (or output) a probability distribution across SAFE tokens (e.g., a token vocabulary as described above) to represent a predicted probability for each SAFE token being the SAFE token that completes a partial SAFE string representation (or fills a subsequent token). Indeed, in some cases, the generative model generates a vector (or array) having a size representative of a token vocabulary with a predicted probability for each token in a token vocabulary being the predicted SAFE token for the partial SAFE string representation (or the next or subsequent SAFE token). In addition, the probability distribution can also include special tokens (e.g., end-of-sequence tokens, beginning-of-sequence tokens) to indicate a probability of the predicted token being an end of sequence (e.g., a prediction that a valid molecule is represented by the predicted sequence of SAFE tokens). In one or more implementations, the digital molecular structure generation system 1306 utilizes the SAFE generative model to generate (or learn) a token distribution utilizing batch training (e.g., of multiple training SAFE string representations) (to train for past sequence context across multiple training SAFE string representations).

In addition, the digital molecular structure generation system 1306 can utilize predicted token distributions (e.g., as the predicted sequence of the SAFE molecular string representation) and a ground truth training SAFE molecular string representation sequence of tokens to determine whether the predicted token distributions correctly (or incorrectly) indicate the correct ground truth token. Indeed, the digital molecular structure generation system 1306 can utilize the comparison to generate a measure of loss that rewards and/or penalizes the generative model (e.g., the large language model 412) based on the token probability distribution indicating the correct or incorrect predicted SAFE token.

Moreover, the digital molecular structure generation system 1306 can utilize various types of losses to train a SAFE generative model (e.g., the large language model 412). For instance, in some cases, the digital molecular structure generation system 1306 generates a cross-entropy measure of loss as the measure of loss (e.g., the measure of loss 414). In one or more instances, the digital molecular structure generation system 1306 can also utilize a variety of other loss measures, such as, but not limited to, mean-squared error losses and/or negative log-likelihood (NLL) losses.

In some embodiments, the digital molecular structure generation system 1306 can fine-tune a SAFE generative model on a specialized chemical space (e.g., for target tasks). For instance, the digital molecular structure generation system 1306 can fine-tune a SAFE generative model for a particular drug utilizing a fragment-constrained target. Furthermore, in some cases, the digital molecular structure generation system 1306 can fine-tune a SAFE generative model for multi-property optimization (MPO) scenarios, including the integration of a prediction head into the SAFE generative model architecture for simultaneous molecular generation and property prediction.

Although one or more embodiments illustrate the digital molecular structure generation system 1306 utilizing tokens, the digital molecular structure generation system 1306 can utilize string representations (e.g., masked string representations as partial sequences) to train a SAFE generative model in accordance with one or more implementations herein.

Indeed, the digital molecular structure generation system 1306 can train a generative model (e.g., a large language model) to create (or complete) SAFE molecular string representations that represent a valid molecular compound. For instance, as mentioned above, the digital molecular structure generation system 1306 can utilize a SAFE generative model (trained in accordance with one or more implementations herein) to perform a variety of downstream fragment-based molecular design tasks, such as, but not limited to, de novo molecular compound generation tasks, scaffold decoration and motif extension tasks, linker design and scaffold morphing tasks, and/or molecular superstructure generation tasks.

For example, FIG. 5 illustrates the digital molecular structure generation system 1306 utilizing a SAFE generative model to perform various downstream fragment-based molecular design tasks. As shown in FIG. 5, the digital molecular structure generation system 1306 utilizes a SAFE large language model 502 with a molecular design task input(s) 504 to generate a SAFE molecular string representation 520. In some cases, the digital molecular structure generation system 1306 can also receive a request prompt 500 (from a user) with the molecular design task input(s) 504 to generate the SAFE molecular string representation 520. As shown in FIG. 5, the digital molecular structure generation system 1306 utilizes the SAFE large language model 502 to utilize the molecular design task input(s) 504 (and the request prompt 500) to perform the molecular design task by generating and/or completing the SAFE molecular string representation 520.

For instance, the molecular design task input(s) 504 can include a linker generation task 508 and/or a scaffolding morphing tasks 510. The digital molecular structure generation system 1306 can utilize the inputs of the linker generation task 508 and/or a scaffolding morphing task 510 to complete a SAFE molecular compound sequence representation from a partial (or incomplete) SAFE molecular compound sequence in the linker generation task 508 and/or the scaffolding morphing tasks 510. Indeed, the digital molecular structure generation system 1306 can complete the SAFE molecular compound sequence representation (as shown in the linker generation task 508 and/or the scaffolding morphing tasks 510) to generate the SAFE molecular string representation 520.

In particular, the digital molecular structure generation system 1306 can utilize a SAFE generative mode to perform linker generation tasks and/or scaffolding morphing tasks as sequence completion tasks. For instance, the digital molecular structure generation system 1306 can utilize input fragments (in a SAFE string) with a request to link the fragments as an initial sequence for the SAFE generative model. Subsequently, the SAFE generative model can generate a predicted tokens for the missing linker in the input fragments to generate (or complete) a SAFE molecular string representation. Indeed, the digital molecular structure generation system 1306 can utilize the SAFE generative model (trained in accordance with one or more implementations herein) to perform a sequential completion because the order of fragments in a SAFE molecular string representation doesn't affect the underlying molecular graph (e.g., the linked fragments are order agnostic).

In some cases, the digital molecular structure generation system 1306 can utilize a constrained beam search to perform a linker generation task (with a SAFE generative model as described above). For instance, the digital molecular structure generation system 1306 can utilize a constrained beam search to ensure the presence of each fragment (of a molecular compound) in a final molecular representation. During a scaffold morphing task, the digital molecular structure generation system 1306 can generate new molecular representations (or new molecules) for one or more fragments with connectivity constraints (after which the scaffold is inferred and linked to other fragments).

In addition, as shown in FIG. 5, the molecular design task input(s) 504 can include a motif extension task 512 and/or a scaffold decoration task 514. As shown in FIG. 5, the digital molecular structure generation system 1306 can utilize the inputs of the motif extension task 512 and/or the scaffold decoration task 514 to generate new fragments utilizing a SAFE molecular string representation (from an input initial sequence). As shown in FIG. 5, the digital molecular structure generation system 1306 can complete an input initial sequence (from the motif extension task 512 and/or the scaffold decoration task 514) to generate the SAFE molecular string representation 520.

For instance, the digital molecular structure generation system 1306 can utilize the SAFE generative model to frame the motif extension task and/or the scaffold decoration task as a sequential completion task by predicting a new token to generate novel fragments using the SAFE molecular string representation. Indeed, the digital molecular structure generation system 1306 can begin with an initial sequence corresponding to a scaffold or motif (e.g., as shown in the input motif extension task 512 and/or the scaffold decoration task 514) with marked attachment points to predict fragments to add to generate a completed SAFE molecular string representation that represents a valid (novel or known) molecular compound.

Additionally, as shown in FIG. 5, the molecular design task input(s) 504 can include a de novo generation task 516. In particular, as shown in FIG. 5, the digital molecular structure generation system 1306 can utilize a null input as indicated in the de novo generation task 516 (e.g., with a request prompt requesting a new molecular compound sequence) to generate the SAFE molecular string representation 520. Indeed, the digital molecular structure generation system 1306 can sample a new sequence from a learned token distribution generated by the SAFE generative model (in accordance with one or more implementations herein). For instance, the digital molecular structure generation system 1306 can utilize the SAFE generative model to generate a (novel or existing) a SAFE molecular string representation that represents a molecular compound (e.g., via autoregressive generation from learned token distributions of the SAFE generative model).

Furthermore, as shown in FIG. 5, the molecular design task input(s) 504 can include a superstructure generation task 518. For instance, the digital molecular structure generation system 1306 can identify a specified substructure constraint (e.g., an input target molecule compound constraint(s) 506). In addition, the digital molecular structure generation system 1306 can utilize the identified specified substructure constraint with the SAFE generative model to generate attachment points on the substructure to create one or more scaffolds. Subsequently, the digital molecular structure generation system 1306 can utilize the SAFE generative model to perform a scaffold decoration task (as described above) on the specified substructure with the generated attachment points to generate the SAFE molecular string representation 520. Indeed, the digital molecular structure generation system 1306 can generate (novel or existing) molecule compound representations (as SAFE representations) that adhere to specified substructure constraints by utilizing an input superstructure generation task with one or more target molecule compound constraint(s).

As also shown in FIG. 5, the digital molecular structure generation system 1306 can also receive one or more target molecule compound constraint(s) 506 (e.g., as part of the request prompt 500). Indeed, the target molecule compound constraint(s) 506 can include a constraint condition to instruct the SAFE generative model to generate a SAFE molecular string representation to represent a molecular compound with the specified constraint(s). For instance, the digital molecular structure generation system 1306 can utilize target molecule compound constraint(s) 506 that include constraint conditions that require particular target fragment(s) in a generated SAFE molecular string representation (e.g., generate a molecular compound that includes a nitrogen atom, generate a molecular compound that includes nitrogen and hydrogen). In some cases, the target molecule compound constraint(s) 506 can include constraints that specify one or more attachment points at which to generate new fragments or particular fragments to add at one or more specified attachment points. In some implementations, the target molecule compound constraint(s) 506 can include instructions to generate a particular link or attachment points between SAFE representations to cause the SAFE generative model to generate an output SAFE molecular string representation that creates a fragment link (or ring link character) as specified by the molecule compound constraint(s) 506. Indeed, the digital molecular structure generation system 1306 can utilize a variety of (or combinations of) the target molecule compound constraints with the SAFE generative model to perform one or more of the downstream fragment-based molecular design tasks (e.g., de novo molecular compound generation tasks, scaffold decoration and motif extension tasks, linker design and scaffold morphing tasks, and/or molecular superstructure generation tasks).

Furthermore, the digital molecular structure generation system 1306 can utilize a target molecular compound constraint(s) 506 to optimize a SAFE generative model to fit one or more of the target profiles defined by the target molecular compound constraint(s) 506. In some cases, the digital molecular structure generation system 1306 can generate a set (or library) of molecule compounds using SAFE representations from the SAFE generative model using a target molecular compound constraint(s) 506.

In some instances, the digital molecular structure generation system 1306 can utilize a SAFE generative model (for downstream fragment-based molecular design tasks) using SMILES (or other molecular string representation) inputs. For instance, the digital molecular structure generation system 1306 can convert the SMILES (or other molecular string representation) inputs into SAFE representations (in accordance with one or more implementations herein). Then, the digital molecular structure generation system 1306 can utilize the converted SAFE representations as inputs for the SAFE generative model to accomplish a downstream fragment-based molecular design task (in accordance with one or more implementations herein).

In one or more instances, the digital molecular structure generation system 1306 can utilize a SAFE generative model (as described herein) and/or a SAFE molecular string representation (as described herein) for a molecule compound with a variety of tech-bio exploration tools of the tech-bio exploration system 1304. For instance, the digital molecular structure generation system 1306 can utilize the SAFE generative model (as described herein) and/or a SAFE molecular string representation to provide (and/or generate) molecule compounds and utilize the molecule compounds as input (or as a component) of the variety of tech-bio exploration tools of the tech-bio exploration system 1304. For instance, the digital molecular structure generation system 1306 can utilize the SAFE generative model (as described herein) and/or a SAFE molecular string representation (as described herein) for tech-bio exploration tools, such as, but not limited to, bio-activity heatmap models as described in UTILIZING MACHINE LEARNING MODELS TO SYNTHESIZE PERTURBATION DATA TO GENERATE PERTURBATION HEATMAP GRAPHICAL USER INTERFACES, U.S. patent application Ser. No. 18/526,707, filed Dec. 1, 2023, ADMET prediction models and/or drug-likeness matching tools as described in UTILIZING COMPOUND-PROTEIN MACHINE LEARNING REPRESENTATIONS TO GENERATE BIOACTIVITY PREDICTIONS, U.S. patent application Ser. No. 18/505,728, filed Nov. 9, 2023, compound exploration program models as described in UTILIZING BIOLOGICAL MACHINE LEARNING REPRESENTATIONS AND A LANGUAGE MACHINE LEARNING MODEL FOR INITIATING COMPOUND EXPLORATION PROGRAMS, U.S. patent application Ser. No. 18/521,910, filed Nov. 28, 2023, digital maps of biology models as described in UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, U.S. patent application Ser. No. 18/392,989, filed Dec. 21, 2023, and/or microscopy representation autoencoder models as described in UTILIZING MASKED AUTOENCODER GENERATIVE MODELS TO EXTRACT MICROSCOPY REPRESENTATION AUTOENCODER EMBEDDINGS, U.S. patent application Ser. No. 18/545,399, filed Dec. 19, 2023, each of which are incorporated by reference in their entirety herein.

In some instances, the digital molecular structure generation system 1306 can identify a molecular compound of interest (e.g., a molecular compound from a compound exploration program as described in U.S. patent application Ser. No. 18/521,910 (incorporated by reference above)). Moreover, the digital molecular structure generation system 1306 can utilize the molecular compound of interest as a constraint (e.g., a target molecule compound constraint) for the SAFE generative model to cause the SAFE generative model to generate molecule compound representations (e.g., SAFE representations) that are related to (or variations) of the molecular compound of interest. In some cases, the digital molecular structure generation system 1306 can identify a database of particular compounds (e.g., enamines, amines) and utilize the SAFE generative model to generate (or synthesize) molecule compounds based on the database of particular compounds as a target molecule compound constraint.

Although one or more particular tech-bio exploration tools are described above, in one or more instances, the digital molecular structure generation system 1306 can also enable the SAFE generative model (or SAFE molecular string representations) to interact with a variety of other tech-bio exploration tools. In addition, the digital molecular structure generation system 1306 can also enable the SAFE generative model (or SAFE molecular string representations) to interact with a variety of third-party (or external) tools, such as, third-party vendor systems, third-party automated lab tools, and/or third-party image editing tools.

Furthermore, experimenters utilized an implementation of a SAFE generative model of the digital molecular structure generation system 1306 (as described above) to generate sample output SAFE molecular representations for a variety of tasks (e.g., linker design, scaffold morphing, motif extension, scaffold decoration, superstructure) for fragment-constrained inputs based on a particular molecular compound (representing the drug Maribavir). For instance, FIG. 6 illustrates the generated output SAFE molecular representations for the variety of tasks (e.g., linker design, scaffold morphing, motif extension, scaffold decoration, superstructure) in response to fragment-constrained inputs based on the particular molecular compound (using an implementation of the SAFE generative model).

Additionally, experimenters examined an implementation of a SAFE generative model's (of the digital molecular structure generation system 1306 as described above) ability to perform fragment-constrained generative design tasks, such as scaffold decoration, scaffold morphing, linker generation, motif extension, and superstructure generation. Indeed, the experimenters designed a benchmark that involved working with scaffolds and fragments from 10 existing drugs to demonstrate the accuracy of the implementation of the SAFE generative model (via validity, diversity, uniqueness, distance, and synthetic accessibility scores). Indeed, the experimenters utilized a 1000 molecules sampled in each of the above-mentioned fragment-constrained design task using an implementation of the SAFE generative model to determine averaged validity, diversity, and uniqueness scores for the outputs of the SAFE generative model. In addition, the experimenters also determined an average Tanimoto distance between the generated molecules to the original drug molecules, along with the average synthetic accessibility (SA) scores (as described in Ertl et al., Estimation of Synthetic Accessibility Score of Drug-Like Molecules Based on Molecular Complexity and Fragment Contributions, Journal of Cheminformatics, 1:1-11 (2009) (hereinafter “Ertr”)). As shown in the following Table (e.g., Table 1), the implementation of the SAFE generative model maintained full validity for the sampled molecules under constraints, while achieving high internal diversity and novelty compared to the original drugs. Moreover, as shown in Table 1, the generated molecules exhibited a low SA score, indicating their ease of synthesis.

TABLE 1

| Task | Validity ↑ | Diversity ↑ | Uniqueness ↑ | Distance ↑ | SA Score ↑ |
|---|---|---|---|---|---|
| Linker Design | 1.000 ± 0.000 | 0.641 ± 0.099 | 0.887 ± 0.191 | 0.712 ± 0.097 | 3.864 ± 0.928 |
| Motif Extension | 1.000 ± 0.000 | 0.681 ± 0.089 | 0.923 ± 0.179 | 0.772 ± 0.101 | 3.750 ± 0.651 |
| Scaffold Decoration | 1.000 ± 0.000 | 0.571 ± 0.113 | 0.851 ± 0.162 | 0.643 ± 0.137 | 4.017 ± 0.889 |
| Scaffold Morphing | 1.000 ± 0.000 | 0.608 ± 0.096 | 0.717 ± 0.219 | 0.688 ± 0.113 | 3.604 ± 0.910 |
| Superstructure | 1.000 ± 0.000 | 0.715 ± 0.059 | 0.929 ± 0.106 | 0.812 ± 0.063 | 3.868 ± 0.919 |

Furthermore, the experimenters also evaluated an implementation of the SAFE generative model in goal-directed generation. For instance, the experimenters optimized an implementation of the SAFE generative model toward specific values for key molecular properties to assess the model's ability for goal-directed generation. For instance, the experimenters optimized towards specific values for molecular properties, including Topological Polar Surface Area (TPSA), Molecular Weight (MW), Calculated Log P (C LOG P), and Quantitative Estimation of Drug-likeness (QED). Indeed, the experimenters utilized an implementation of the SAFE generative model using Proximal Policy Optimization (PPO) (as described in Schulman et al., Proximal Policy Optimization Algorithms, arXiv Preprint arXiv:1707.06347 (2017)(hereinafter “Schulman”)) with Adaptive KL Penalty to train a policy for generating molecular samples with the targeted property value. The experimenters further fine-tuned agents (of an implementation of the SAFE generative model) for two target values on each molecular property and evaluated their performance. Indeed, the generated samples, from the experiment, were valid and unique.

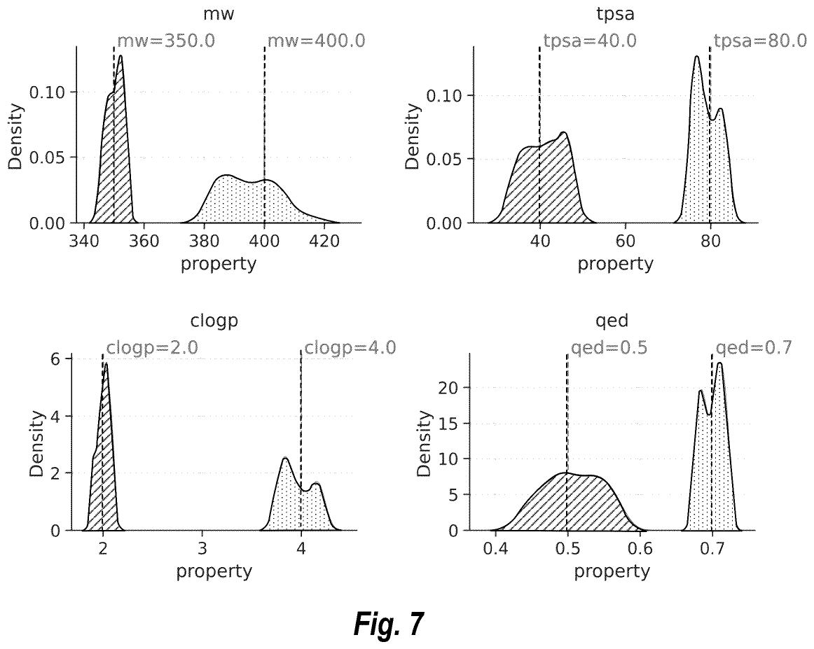

Indeed, FIG. 7 illustrates a property distribution of the generated samples from the above-described experiment. Indeed, the dashed vertical line, in the charts illustrated on FIG. 7, represents the target value of the molecular property that the implementation of the SAFE generative model was optimized towards. In addition, the dashed and dotted histograms represent the distribution of samples from different implementations of the SAFE generative model having distinct molecular property goals. In particular, the results depicted in FIG. 7 demonstrate that the property distribution of the generated molecules, achieved through goal-conditioned optimization using PPO on implementations of the SAFE generative models, is notably centered around the respective target values. This indicates the success of the optimization process' on the implementations of the SAFE generative models aligning the generated molecules distribution with the desired property targets.

Additionally, experimenters also utilized an implementation of the SAFE generative model on an optimization task aimed at improving the Central Nervous System (CNS) penetration of EGFR Tyrosine Kinase Inhibitors (e.g., addressing the challenge of CNS metastases in non-small cell lung cancer). Indeed, the experimenters evaluated for a CNS-MPO score, a comprehensive metric that assesses physico-chemical properties associated with CNS penetration (with a higher CNS-MPO score indicating better desirability). In addition, the experimenters introduced additional constraints to our optimization task which required that generated molecules feature a scaffold that has demonstrated activity against EGFR.

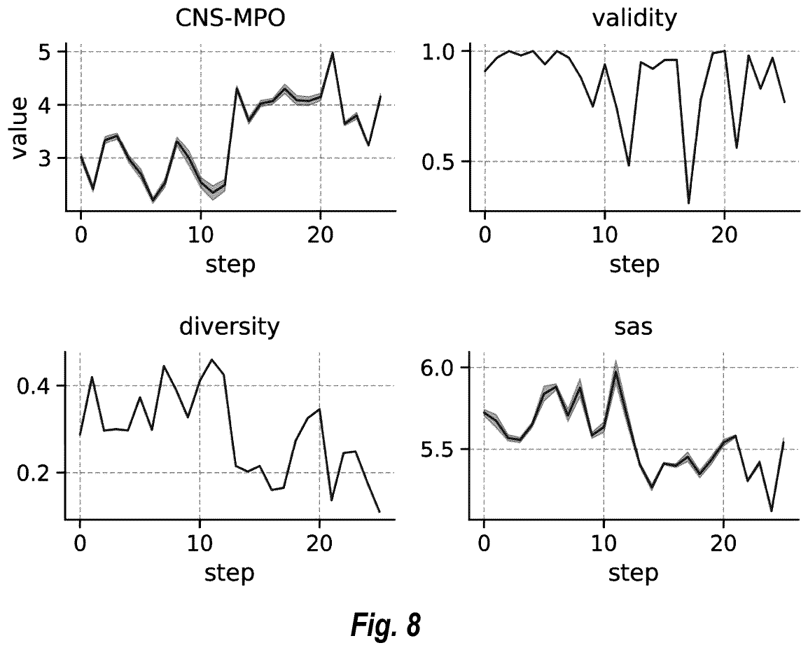

Indeed, FIG. 8 illustrates a reward distribution obtained by sampling 100 molecules at each optimization iteration (e.g., 25 steps) of the implementation of the SAFE generative model. As shown in FIG. 8, the implementation of the SAFE generative model is capable of efficiently executing scaffold-constrained optimization using a straightforward optimization algorithm, such as PPO. Indeed, as shown in FIG. 8, the implementation of the SAFE generative model optimized to improve CNS penetration result in a reduction in the diversity of sampled candidates while overall validity remains robust. In addition, FIG. 8 also illustrates a decline in the SA score across iterations, indicating the presence of synthetic feasibility of the molecule representations generated by the SAFE generative model.

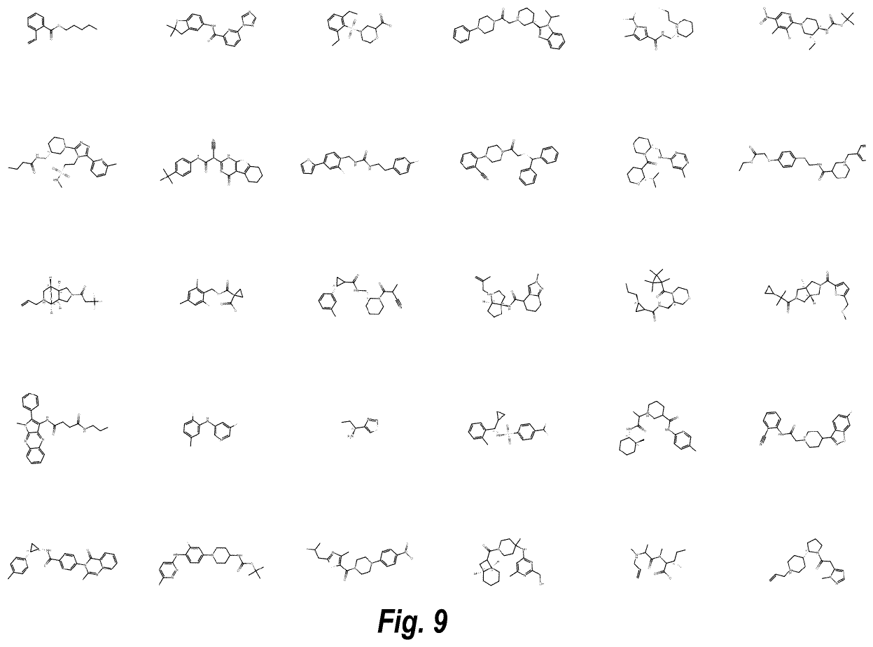

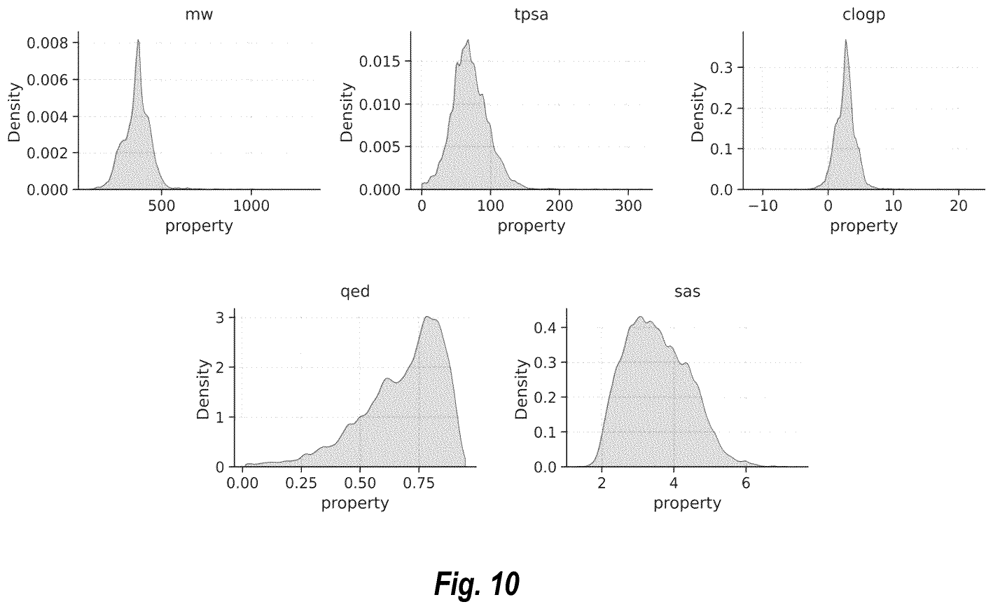

Furthermore, experimenters utilized an implementation of the SAFE generative model to generate de novo molecule representations. Indeed, FIG. 9 illustrates randomly selected samples of de novo generated molecules utilizing an implementation of the SAFE generative model. Additionally, the experimenters also evaluated a molecular property distribution for 10,000 molecules generated utilizing an implementation of the SAFE generative model (e.g., for properties, such as MW, TPSA, C LOG P, QED, and SA scores). Indeed, FIG. 10 illustrates the molecular property distribution for the 10,000 molecules generated utilizing the implementation of the SAFE generative model. As shown in FIG. 10, the property distribution demonstrates that the implementation of the SAFE generative model can generate molecules with diverse physicochemical properties spanning beyond traditional drug-like molecules.

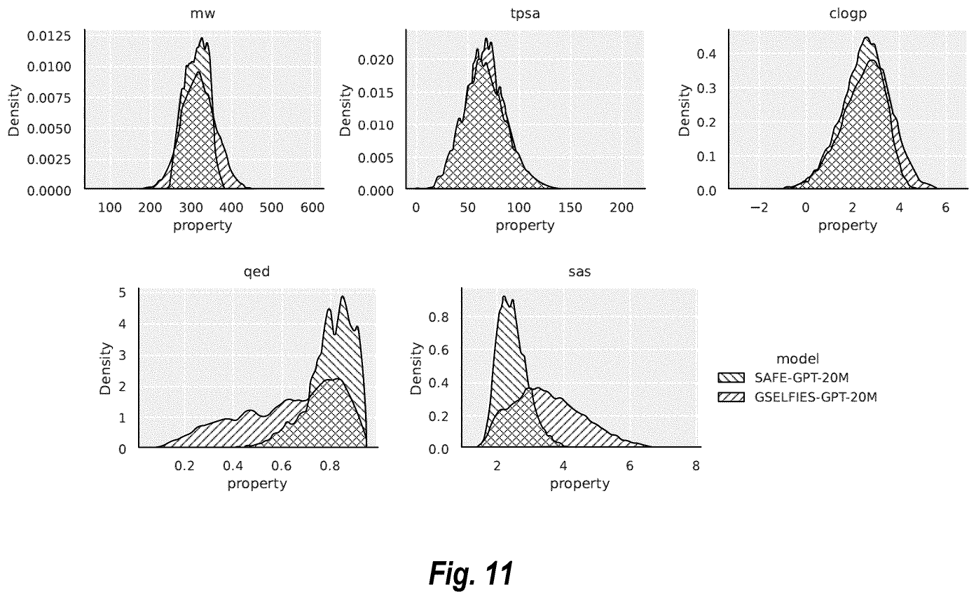

Furthermore, the experimenters also compared de novo molecule generation between an implementation of the SAFE generative model (e.g., SAFE-GPT-20M) and a Group SELFLIES model (GSELFIES-GPT-20M), both trained on a MOSES dataset). Indeed, FIG. 11 illustrates a property distribution of 10,000 molecules generated on the SAFE-GPT-20M and GSELFIES-GPT-20M models. As shown in FIG. 11, the molecules generated by SAFE-GPT-20M exhibit higher QED scores (which indicates a higher degree of drug-likeness) and lower SA scores (which indicates better synthetic feasibility).

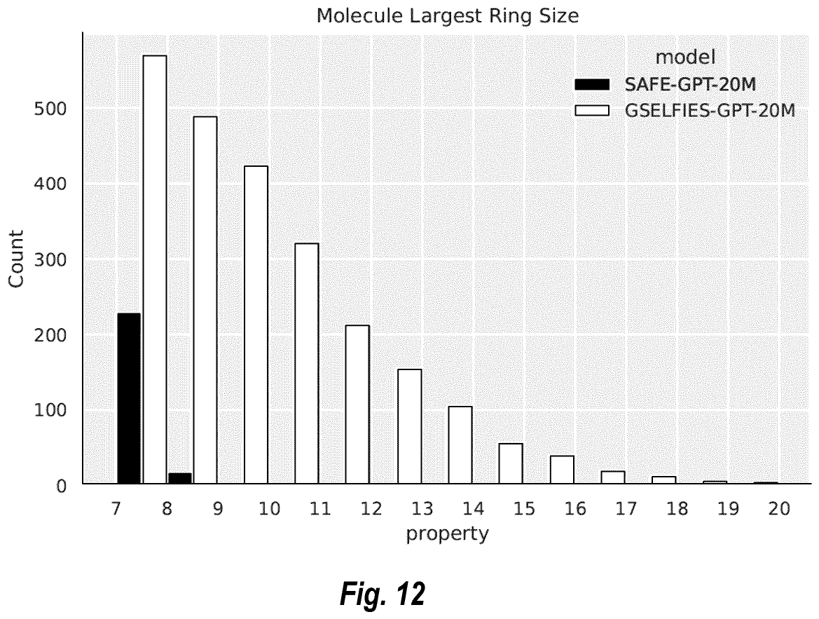

Additionally, as mentioned above, existing systems that utilizes GSELFIES representations lack simplicity, are difficult to interpret, and are not compact. In contrast, experimenters demonstrate that an implementation of the SAFE generative model (e.g., the SAFE-GPT-20M described above) generates SAFE representations that are compact and easier to interpret. For instance, FIG. 12 illustrates a comparison of distributions of ring size of molecules generated by the SAFE-GPT-20M and GSELFIES-GPT-20M models. As shown in FIG. 12, the GSELFIES-GPT-20M model frequently generates molecules with large and unstable ring structures (e.g., non-druglike structures) and the SAFE-GPT-20M model generates molecule representations with less ring sizes indicating drug-likeness and improved structure stability.

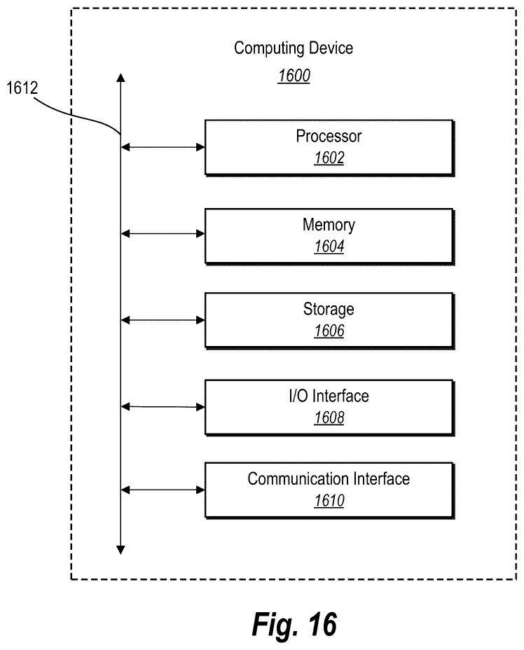

FIG. 13 illustrates a schematic diagram of a system environment in which the digital molecular structure generation system 1306 can operate in accordance with one or more embodiments. As shown in FIG. 13, the environment includes server(s) 1302 (which includes a tech-bio exploration system 1304 and the digital molecular structure generation system 1306), a network 1308, client device(s) 1310, and testing device(s) 1312. As further illustrated in FIG. 13, the various computing devices within the environment can communicate via the network 1308. Although FIG. 13 illustrates the digital molecular structure generation system 1306 being implemented by a particular component and/or device within the environment, the digital molecular structure generation system 1306 can be implemented, in whole or in part, by other computing devices and/or components in the environment (e.g., the client device(s) 1310). Additional description regarding the illustrated computing devices is provided with respect to FIG. 16 below.

As shown in FIG. 13, the server(s) 1302 can include the tech-bio exploration system 1304. In some embodiments, the tech-bio exploration system 1304 can determine, store, generate, and/or display tech-bio information including molecular compounds (or molecular compound string representations), maps of biology, biology experiments from various sources, and/or machine learning tech-bio predictions. For instance, the tech-bio exploration system 1304 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal of in-vivo experiments involving chemical compounds). In one or more embodiments, the server(s) 1302 comprises a data server. In some implementations, the server(s) 1302 comprises a communication server or a web-hosting server.

For instance, the tech-bio exploration system 1304 can generate and access experimental results corresponding to gene sequences, protein shapes/folding, protein/compound interactions, phenotypes resulting from various interventions or perturbations (e.g., gene knockout sequences or compound treatments), and/or in-vivo experimentation on various treatments in living animals. By analyzing these signals (e.g., utilizing various machine learning models), the tech-bio exploration system 1304 can generate or determine a variety of predictions and inter-relationships for improving treatments/interventions.

To illustrate, the tech-bio exploration system 1304 can generate maps of biology indicating biological inter-relationships or similarities between these various input signals to discover potential new treatments. For example, the tech-bio exploration system 1304 can utilize machine learning and/or maps of biology to identify a similarity between a first gene associated with disease treatment and a second gene previously unassociated with the disease based on a similarity in resulting phenotypes from gene knockout experiments. The tech-bio exploration system 1304 can then identify new treatments based on the gene similarity (e.g., by targeting molecular compounds the impact the second gene). Similarly, the tech-bio exploration system 1304 can analyze signals from a variety of sources (e.g., protein interactions, or in-vivo experiments) to predict efficacious treatments based on various levels of biological data.

The tech-bio exploration system 1304 can generate GUIs comprising dynamic user interface elements to convey tech-bio information and receive user input for intelligently exploring tech-bio information. Indeed, as mentioned above, the tech-bio exploration system 1304 can generate GUIs displaying different maps of biology that intuitively and efficiently express complex interactions between different biological systems for identifying improved treatment solutions. Furthermore, the tech-bio exploration system 1304 can also electronically communicate tech-bio information between various computing devices.

As shown in FIG. 13, the tech-bio exploration system 1304 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1304 collects, manages, and transmits data across a variety of different entities, accounts, and devices. In some cases, the tech-bio exploration system 1304 is a network system that facilitates access to (and analysis of) tech-bio information within a centralized operating system. Indeed, the tech-bio exploration system 1304 can link data from different network-based research institutions to generate and analyze maps of biology.

As shown in FIG. 13, the tech-bio exploration system 1304 can include a system that comprises the digital molecular structure generation system 1306 that can generate one or more SAFE molecular representations (or train SAFE generative models) in accordance with one or more implementations herein. For example, digital molecular structure generation system 1306 can convert molecular representations to SAFE molecular representations as described above. In addition, the digital molecular structure generation system 1306 can utilize SAFE molecular representations to train SAFE generative models (e.g., large language models) to perform a variety of downstream molecular design tasks (as described above).

As also illustrated in FIG. 13, the environment includes the client device(s) 1310. For example, the client device(s) 1310 may include, but is not limited to, a mobile device (e.g., smartphone, tablet) or other type of computing device, including those explained below with reference to FIG. 16. Additionally, the client device(s) 1310 can include a computing device associated with (and/or operated by) user accounts for the tech-bio exploration system 1304. Moreover, the environment can include various numbers of client devices that communicate and/or interact with the tech-bio exploration system 1304 and/or the digital molecular structure generation system 1306.

Furthermore, in one or more implementations, the client device(s) 1310 includes a client application. The client application can include instructions that (upon execution) cause the client device(s) 1310 to perform various actions. For example, a user of a user account can interact with the client application on the client device(s) 1310 to initiate, generate, or access one or more SAFE molecular representations or SAFE generative models (e.g., via prompts) in accordance with one or more implementations herein.

As further shown in FIG. 13, the environment includes the network 1308. As mentioned above, the network 1308 can enable communication between components of the environment. In one or more embodiments, the network 1308 may include a suitable network and may communicate using a various number of communication platforms and technologies suitable for transmitting data and/or communication signals, examples of which are described with reference to FIG. 16. Furthermore, although FIG. 13 illustrates computing devices communicating via the network 1308, the various components of the environment can communicate and/or interact via other methods (e.g., communicate directly).

In one or more implementations, the digital molecular structure generation system 1306 generates and accesses one or more SAFE molecular representations and/or SAFE generative models. As shown, in FIG. 13, the digital molecular structure generation system 1306 can communicate with testing device(s) 1312 to utilize, obtain, analyze, generate, and/or store this information. For example, the tech-bio exploration system 1304 can interact with the testing device(s) 1312 that include intelligent robotic devices and camera devices for generating and capturing digital images of cellular phenotypes resulting from different perturbations (e.g., genetic knockouts or compound treatments of stem cells). Similarly, the testing device(s) 1312 can include camera devices and/or other sensors (e.g., heat or motion sensors) capturing real-time information from animals as part of in-vivo experimentation (e.g., biomarker data). The tech-bio exploration system 1304 can also interact with a variety of other testing device(s) such as devices for determining, generating, or extracting gene sequences or protein information.

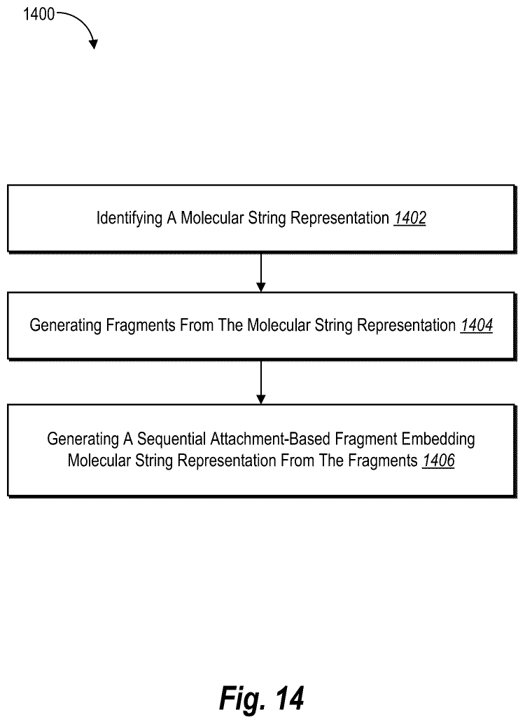

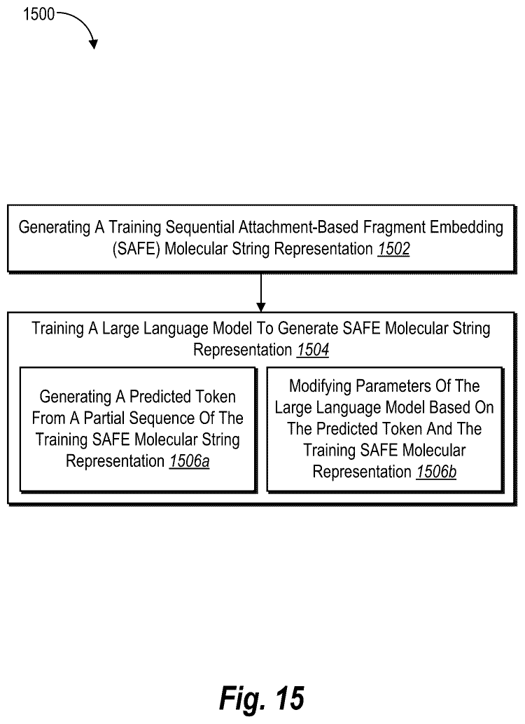

FIGS. 1-13, the corresponding text, and the examples provide a number of different systems, computer-implemented methods, and non-transitory computer readable media for generating SAFE molecular representations in accordance with one or more implementations herein. In addition to the foregoing, embodiments can also be described in terms of flowcharts comprising acts for accomplishing a particular result. For example, FIGS. 14 and 15 illustrate flowcharts of example sequences of acts in accordance with one or more embodiments.

While FIGS. 14 and/or 15 illustrates acts according to some embodiments, alternative embodiments may omit, add to, reorder, and/or modify any of the acts shown in FIGS. 14 and/or 15. The acts of FIGS. 14 and/or 15 can be performed as part of a (computer-implemented) method. Alternatively, a non-transitory computer readable medium can comprise instructions, that when executed by one or more processors, cause a computing device to perform the acts of FIGS. 14 and/or 15. In still further embodiments, a system can perform the acts of FIGS. 14 and/or 15. Additionally, the acts described herein may be repeated or performed in parallel with one another or in parallel with different instances of the same or other similar acts.

For instance, FIG. 14 illustrates an example series of acts for generating a SAFE molecular representation in accordance with one or more embodiments. For example, as shown in FIG. 14, the series of acts 1400 can include an act 1402 of identifying a molecular string representation, an act 1404 of generating fragments from the molecular string representation, and an act 1406 of generating a sequential attachment-based fragment embedding molecular string representation from the fragments.

In one or more instances, the series of acts 1400 can include identifying a molecular string representation comprising ring structure identifiers that indicate virtual connections between atom representations of a molecular compound, generating a set of fragments from the molecular string representation, and generating a sequential attachment-based fragment embedding (SAFE) molecular string representation that represents the molecular string representation as an order agnostic sequence of interconnected fragment blocks by: concatenating fragments from the set of fragments utilizing a separation character between the fragments to generate a linked fragment string and generating ring link characters in the linked fragment string to represent attachment points for fragment links.

Furthermore, the series of acts 1400 can include generating the set of fragments by utilizing a bond slicing algorithm with the molecular string representation. In addition, the series of acts 1400 can include generating the linked fragment string by ordering the fragments from the set of fragments based on fragment size.

In addition, the series of acts 1400 can include generating the SAFE molecular string representation by extracting attachment point indicators from the molecular string representation and utilizing the attachment point indicators to generate the linked fragment string. Moreover, the series of acts 1400 can include generating the SAFE molecular string representation by replacing the attachment point indicators in the linked fragment string with the ring link characters.

Furthermore, the series of acts 1400 can include generating an additional SAFE molecular string representation from the SAFE molecular string representation by reordering fragment blocks comprising the fragments and the ring link characters, wherein the additional SAFE molecular string representation represents the molecular string representation. For example, a ring link character(s) can include a ring digit(s).

Moreover, the series of acts 1400 can include generating, utilizing a large language model from the SAFE molecular string representation, an additional SAFE molecular string representation representing an additional molecular compound. In addition, the series of acts 1400 can include generating, utilizing a large language model from the SAFE molecular string representation, a complete SAFE molecular compound sequence representation from a partial SAFE molecular compound sequence representation. Furthermore, the series of acts 1400 can include generating, utilizing a large language model from the SAFE molecular string representation, a linking SAFE molecular string representation for two or more molecular compound sequences. Additionally, the series of acts 1400 can include generating, utilizing a large language model from the SAFE molecular string representation, a molecular compound sequence based on one or more target molecule compound constraints.

Furthermore, FIG. 15 illustrates an example series for training a large language model to generate a sequential attachment-based fragment embedding (SAFE) molecular string representation in accordance with one or more embodiments. For instance, as shown in FIG. 15, the series of acts 1500 can include an act 1502 of generating a training sequential attachment-based fragment embedding (SAFE) molecular string representation and an act 1504 of training a large language model to generate a SAFE molecular string representation which includes an act 1506a of generating a predicted token from a partial sequence of the training SAFE molecular string representation and an act 1506b of modifying parameters of the large language model based on the predicted token and the training SAFE molecular representation.

In some implementations, the series of acts 1500 include generating, for a molecular compound, a training sequential attachment-based fragment embedding (SAFE) molecular string representation comprising order agnostic fragment blocks represented by fragment strings, separation characters, and ring link characters and training a large language model to generate SAFE molecular string representations by: generating, utilizing the large language model, a predicted token for the training SAFE molecular string representation from a tokenized partial sequence of the training SAFE molecular string representation and modifying parameters of the large language model utilizing a comparison between the predicted token and the training SAFE molecular string representation.

Furthermore, the series of acts 1500 can include generating the training SAFE molecular string representation by converting a molecular string representation comprising ring structure identifiers that indicate virtual connections between atom representations of the molecular compound. In addition, the series of acts 1500 can include generating the training SAFE molecular string representation by concatenating fragments identified from the molecular string representation utilizing the separation characters and representing attachment points for fragment links of the fragments utilizing the ring link character.

Additionally, the series of acts 1500 can include generating, utilizing the large language model, the predicted token by utilizing the large language model to generate a SAFE notation token probability distribution and/or selecting the predicted token from the SAFE notation token probability distribution.

Moreover, the series of acts 1500 can include training the large language model to generate the SAFE molecular string representations by determining a measure of loss between the predicted token and the training SAFE molecular string representation and/or modifying the parameters of the large language model utilizing the measure of loss.

Furthermore, the series of acts 1500 can include generating, utilizing the large language model, an end-of-sequence token as the predicted token to indicate a predicted completed molecule representation.

Additionally, the series of acts 1500 can include utilizing the large language model to complete a partial molecular compound sequence or generate a linking SAFE molecular string representation for two or more molecular compound sequences. Furthermore, the series of acts 1500 can include generating, utilizing the large language model, a SAFE molecular string representation based on a prompt requesting a target molecular compound.

Embodiments of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some implementations, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 16 illustrates a block diagram of exemplary computing device 1600 (e.g., the server(s) 1302 and/or the client device(s) 1310) that may be configured to perform one or more of the processes described above. One will appreciate that server(s) 1302 and/or the client device(s) 1310 may comprise one or more computing devices such as computing device 1600. As shown by FIG. 16, computing device 1600 can comprise processor 1602, memory 1604, storage device 1606, I/O interface 1608, and communication interface 1610, which may be communicatively coupled by way of communication infrastructure 1612. While an exemplary computing device 1600 is shown in FIG. 16, the components illustrated in FIG. 16 are not intended to be limiting. Additional or alternative components may be used in other implementations. Furthermore, in certain implementations, computing device 1600 can include fewer components than those shown in FIG. 16. Components of computing device 1600 shown in FIG. 16 will now be described in additional detail.

In particular implementations, processor 1602 includes hardware for executing instructions, such as those making up a computer program. As an example and not by way of limitation, to execute instructions, processor 1602 may retrieve (or fetch) the instructions from an internal register, an internal cache, memory 1604, or storage device 1606 and decode and execute them. In particular implementations, processor 1602 may include one or more internal caches for data, instructions, or addresses. As an example and not by way of limitation, processor 1602 may include one or more instruction caches, one or more data caches, and one or more translation lookaside buffers (TLBs). Instructions in the instruction caches may be copies of instructions in memory 1604 or storage device 1606.

Memory 1604 may be used for storing data, metadata, and programs for execution by the processor(s). Memory 1604 may include one or more of volatile and non-volatile memories, such as Random Access Memory (“RAM”), Read Only Memory (“ROM”), a solid state disk (“SSD”), Flash, Phase Change Memory (“PCM”), or other types of data storage. Memory 1604 may be internal or distributed memory.

Storage device 1606 includes storage for storing data or instructions. As an example and not by way of limitation, storage device 1606 can comprise a non-transitory storage medium described above. Storage device 1606 may include a hard disk drive (HDD), a floppy disk drive, flash memory, an optical disc, a magneto-optical disc, magnetic tape, or a Universal Serial Bus (USB) drive or a combination of two or more of these. Storage device 1606 may include removable or non-removable (or fixed) media, where appropriate. Storage device 1606 may be internal or external to computing device 1600. In particular implementations, storage device 1606 is non-volatile, solid-state memory. In other implementations, Storage device 1606 includes read-only memory (ROM). Where appropriate, this ROM may be a mask programmed ROM, programmable ROM (PROM), erasable PROM (EPROM), electrically erasable PROM (EEPROM), electrically alterable ROM (EAROM), or flash memory or a combination of two or more of these.

I/O interface 1608 allows a user to provide input to, receive output from, and otherwise transfer data to and receive data from computing device 1600. I/O interface 1608 may include a mouse, a keypad or a keyboard, a touch screen, a camera, an optical scanner, network interface, modem, other known I/O devices or a combination of such I/O interfaces. I/O interface 1608 may include one or more devices for presenting output to a user, including, but not limited to, a graphics engine, a display (e.g., a display screen), one or more output drivers (e.g., display drivers), one or more audio speakers, and one or more audio drivers. In certain implementations, I/O interface 1608 is configured to provide graphical data to a display for presentation to a user. The graphical data may be representative of one or more graphical user interfaces and/or any other graphical content as may serve a particular implementation.

Communication interface 1610 can include hardware, software, or both. In any event, communication interface 1610 can provide one or more interfaces for communication (such as, for example, packet-based communication) between computing device 1600 and one or more other computing devices or networks. As an example and not by way of limitation, communication interface 1610 may include a network interface controller (NIC) or network adapter for communicating with an Ethernet or other wire-based network or a wireless NIC (WNIC) or wireless adapter for communicating with a wireless network, such as a WI-FI.

Additionally or alternatively, communication interface 1610 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, communication interface 1610 may facilitate communications with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof.

Additionally, communication interface 1610 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

Communication infrastructure 1612 may include hardware, software, or both that couples components of computing device 1600 to each other. As an example and not by way of limitation, communication infrastructure 1612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, for a molecular compound, a training sequential attachment-based fragment embedding (SAFE) molecular string representation comprising order agnostic fragment blocks represented by fragment strings, separation characters, and ring link characters; and
training a large language model to generate SAFE molecular string representations by:
generating, utilizing the large language model, a predicted token for the training SAFE molecular string representation from a tokenized partial sequence of the training SAFE molecular string representation; and
modifying parameters of the large language model utilizing a comparison between the predicted token and the training SAFE molecular string representation.

2. The computer-implemented method of claim 1, further comprising generating the training SAFE molecular string representation by converting a molecular string representation comprising ring structure identifiers that indicate virtual connections between atom representations of the molecular compound.

3. The computer-implemented method of claim 2, further comprising generating the training SAFE molecular string representation by concatenating fragments identified from the molecular string representation utilizing the separation characters and representing attachment points for fragment links of the fragments utilizing the ring link characters.

4. The computer-implemented method of claim 1, further comprising generating, utilizing the large language model, the predicted token by:
utilizing the large language model to generate a SAFE notation token probability distribution; and
selecting the predicted token from the SAFE notation token probability distribution.

5. The computer-implemented method of claim 1, further comprising training the large language model to generate the SAFE molecular string representations by:
determining a measure of loss between the predicted token and the training SAFE molecular string representation; and
modifying the parameters of the large language model utilizing the measure of loss.

6. The computer-implemented method of claim 1, further comprising generating, utilizing the large language model, an end-of-sequence token as the predicted token to indicate a predicted completed molecule representation.

7. The computer-implemented method of claim 1, further comprising utilizing the large language model to complete a partial molecular compound sequence or generate a linking SAFE molecular string representation for two or more molecular compound sequences.

8. The computer-implemented method of claim 1, further comprising generating, utilizing the large language model, a SAFE molecular string representation based on a prompt requesting a target molecular compound.

9. A system comprising:
at least one processor; and
at least one non-transitory computer-readable storage medium storing instructions that, when executed by the at least one processor, cause the system to:
generate, for a molecular compound, a training sequential attachment-based fragment embedding (SAFE) molecular string representation comprising order agnostic fragment blocks represented by fragment strings, separation characters, and ring link characters; and
train a large language model to generate SAFE molecular string representations by:
generating, utilizing the large language model, a predicted token for the training SAFE molecular string representation from a tokenized partial sequence of the training SAFE molecular string representation; and
modifying parameters of the large language model utilizing a comparison between the predicted token and the training SAFE molecular string representation.

10. The system of claim 9, wherein the instructions cause the system to generate the training SAFE molecular string representation by converting a molecular string representation comprising ring structure identifiers that indicate virtual connections between atom representations of the molecular compound.

11. The system of claim 9, wherein the instructions cause the system to generate, utilizing the large language model, the predicted token by:
utilizing the large language model to generate a SAFE notation token probability distribution; and
selecting the predicted token from the SAFE notation token probability distribution.

12. The system of claim 9, wherein the instructions cause the system to train the large language model to generate the SAFE molecular string representations by:
determining a measure of loss between the predicted token and the training SAFE molecular string representation; and
modifying the parameters of the large language model utilizing the measure of loss.

13. The system of claim 9, wherein the instructions cause the system to generate, utilizing the large language model, an end-of-sequence token as the predicted token to indicate a predicted completed molecule representation.

14. The system of claim 9, wherein the instructions cause the system to utilize the large language model to complete a partial molecular compound sequence or generate a linking SAFE molecular string representation for two or more molecular compound sequences.

15. A non-transitory computer-readable medium storing instructions that, when executed by at least one processor, cause a computing device to:
generate, for a molecular compound, a training sequential attachment-based fragment embedding (SAFE) molecular string representation comprising order agnostic fragment blocks represented by fragment strings, separation characters, and ring link characters; and
train a large language model to generate SAFE molecular string representations by:
generating, utilizing the large language model, a predicted token for the training SAFE molecular string representation from a tokenized partial sequence of the training SAFE molecular string representation; and
modifying parameters of the large language model utilizing a comparison between the predicted token and the training SAFE molecular string representation.

16. The non-transitory computer-readable medium of claim 15, wherein the instructions cause the computing device to generating the training SAFE molecular string representation by converting a molecular string representation comprising ring structure identifiers that indicate virtual connections between atom representations of the molecular compound by concatenating fragments identified from the molecular string representation utilizing the separation characters and representing attachment points for fragment links of the fragments utilizing the ring link characters.

17. The non-transitory computer-readable medium of claim 15, wherein the instructions cause the computing device to generate, utilizing the large language model, the predicted token by:
utilizing the large language model to generate a SAFE notation token probability distribution; and
selecting the predicted token from the SAFE notation token probability distribution.

18. The non-transitory computer-readable medium of claim 15, wherein the instructions cause the computing device to train the large language model to generate the SAFE molecular string representations by:
determining a measure of loss between the predicted token and the training SAFE molecular string representation; and
modifying the parameters of the large language model utilizing the measure of loss.

19. The non-transitory computer-readable medium of claim 15, wherein the instructions cause the computing device to generate, utilizing the large language model, an end-of-sequence token as the predicted token to indicate a predicted completed molecule representation.

20. The non-transitory computer-readable medium of claim 15, wherein the instructions cause the computing device to generate, utilizing the large language model, a SAFE molecular string representation based on a prompt requesting a target molecular compound.
